SEQUENCE LISTING (2 sequences)

SEQ ID NO: 1 - protein, 17 aa, Mus musculus
LQVTDSGLYRCVIYHPP

SEQ ID NO: 2 - protein, 17 aa, Mus musculus
TDSRCVIGLYHPPLQVY

(19) United States
(12) Patent Application Publication
(10) Pub. No.: US 2015/0086618 A1
(43) Pub. Date: Mar. 26, 2015

(54) PHOSPHOLIPID MICELLAR AND LIPOSOMAL COMPOSITIONS AND USES THEREOF

(71) Applicant: THE BOARD OF TRUSTEES OF THE UNIVERSITY OF ILLINOIS, URBANA, IL (US)

(72) Inventors: HAYAT ONYUKSEL, WESTERN SPRINGS, IL (US); ISRAEL RUBENSTEIN, HIGHLAND PARK, IL (US)

(21) Appl. No.: 14/502,116

(22) Filed: Sep. 30, 2014

Related U.S. Application Data
(63) Continuation of application No. 13/124,605, filed on Jul. 8, 2011, now abandoned.
(60) Provisional application No. 61/169,215, filed on Apr. 14, 2009.
(60) Provisional application No. 61/167,749, filed on Apr. 8, 2009.
(60) Provisional application No. 61/105,463, filed on Oct. 15, 2008.

(51) Int. Cl. A61K 38/26; A61K 47/24; A61K 9/107

(52) U.S. Cl. CPC A61K 38/26; A61K 9/107; A61K 47/24
USPC 424/450; 514/11.7; 514/1.4; 514/1.5; 514/13.1

(57) ABSTRACT

The invention generally relates to compositions and methods for the reduction or neutralization of toxins associated with a bacterial, mycobacterial, fungal, viral, or protozoal agent. More particularly, the invention is directed to sterically stabilized phospholipid micellar and liposomal compositions, which interact with the toxins to decrease or neutralize their toxicity. Additionally, the invention includes the use of sterically stabilized phospholipid micellar compositions comprising one or more water-insoluble antibiotic, antifungal, antiviral, antiprotozoal, or anti-inflammatory agent(s), wherein the micellar or liposomal composition inhibits the formation of aggregates. The invention further includes the use of sterically stabilized micelle and liposomal compositions to deliver compounds to the site of action, and in some cases targets the compound to the site of action, for the treatment of inflammation and infection. The invention includes the use of combinations of such micellar and liposomal compositions to improve the effectiveness of treatment.

### RELATED APPLICATIONS

This application claims benefit of U.S. Provisional Patent Application Ser. No. 61/105,463, filed Oct. 15, 2008, U.S. Provisional Patent Application Ser. No. 61/167,749, filed Apr. 8, 2009, and U.S. Provisional Patent Application Ser. No. 61/169,215, filed Apr. 14, 2009, each of which is incorporated herein by reference in its entirety.

### GOVERNMENT RIGHTS

This invention was made in part with government support under grant numbers AG024026, CA121797, and CO6RR15482 from the National Institute of Health and VA Merit Review. As such, the United States government has certain rights in the invention.

### FIELD OF THE INVENTION

The invention generally relates to compositions and methods for the reduction or neutralization of toxins associated with a bacterial, mycobacterial, fungal, viral, or protozoal agent. More particularly, the invention is directed to sterically stabilized phospholipid micellar and liposomal compositions, which interact with the toxins to decrease or neutralize their toxicity. In other aspects, the invention is directed to sterically stabilized phospholipid micellar and liposomal compositions, which interact with the toxins to decrease injury in cells and tissues. The invention includes the use of sterically stabilized micelle and liposomal compositions comprising water-insoluble antibiotics, antifungals, antivirals, or antiprotozoal agents and methods for the delivery of such compositions in a subject, wherein the compositions provide increased solubility, increased stability, and decreased toxicity. Even more particularly, the invention includes the use of phospholipid micellar or liposomal compositions in neutralizing bacterial or mycobacterial endotoxins and exotoxins. The invention further includes the use of sterically stabilized micelle and liposomal compositions to deliver compounds to the site of action for the treatment of inflammation and infection. In certain aspects, the invention includes the use of combinations of such micellar and liposomal compositions to improve the effectiveness of treatment.

### BACKGROUND OF THE INVENTION

Liposomes are microscopic spherical structures composed of phospholipids. In aqueous media, phospholipid molecules being amphiphilic spontaneously organize themselves in self-closed bilayers as a result of hydrophilic and hydrophobic interactions. The resulting vesicles, called liposomes, therefore encapsulate in their interior part of the aqueous medium in which they are suspended, a property that makes them potential carriers for biologically active hydrophilic molecules and drugs in vivo.

Sterically stabilized liposomes (SSL) (also known as “PEG-liposomes”) are polymer-coated liposomes, wherein the polymer, in one aspect, polyethylene glycol (PEG), is covalently conjugated to one of the phospholipids and provides a hydrophilic “cloud” outside the vesicle bilayer. This steric barrier delays the recognition by opsonins, allowing SSL to remain in circulation much longer than conventional liposomes and increases the pharmacological efficacy of encapsulated agents. The mechanism by which SSL avoids macrophages and circulate longer in the blood is thought to involve the formation of a “steric barrier” around the liposomes by the attached PEG molecules. The circulation time of SSL may be controlled by selection of their size, PEG molecular weight, chain length and concentration and selection of the lipid composition.

Micelles are colloidal aggregates spontaneously formed by amphiphilic compounds in water above a critical solute concentration, the critical micellar concentration (CMC), and at solution temperatures above the critical micellar temperature (CMT). There are many ways to determine CMC, including surface tension measurements, solubilization of water insoluble dye, or a fluorescent probe, conductivity measurements, light scattering, and the like. For example, surface tension measurements are used to determine the CMC of PEG-DSPE micelles at room temperature.

Surfactant micelles are used as adjuvants and drug carrier systems in many areas of pharmaceutical technology. Micelles have been used to increase bioavailability or decrease adverse effects of drugs (Trubetskoy et al., Advan. Drug Deliv. Reviews 16:311-320 (1995)). In addition, the small size of micelles play a key role in transport across membranes including the blood brain barrier (Muranushi et al., Chemistry and Physics of Lipids 28:269-279 (1981); Saletu et al., Int. Clin. Psychopharmacol. 3:287-323 (1988)). The surfactant micelles are thermodynamically unstable in aqueous media and subject to dissociation upon dilution.

Sterically stabilized phospholipid micelles (SSM) and sterically stabilized mixed micelles (SSMM) are useful as a drug delivery system, especially as therapeutic and diagnostic agents for the delivery of amphiphilic compounds (Onyuksel et al., Pharm. Res. 16(1):155-160 (1999); Ashok et al., J. Pharm. Sci. 93(10):2476-87 (2004); and Koo et al., Nanomedicine 1(3):193-212 (2005)). As Trubetskoy et al. (Proceed. Intern. Symp. Control. Tel. Bioact. Mater. 22:452-453 (1995)) pointed out, almost every possible drug administration route has benefited from the use of micellar drug formation in terms of increased bioavailability or reduced adverse effects.

With an alarming increase in bacterial resistance to antibiotics, there is a need in the art to develop new anti-infective drugs to overcome this phenomenon. To this end, polymyxin B (PxB) is a potent amphiphilic decapeptide antibiotic composed of a hydrophilic polar charged cyclic ring and a hydrophobic 8-carbon acyl chain. PxB is an antibiotic primarily used for resistant Gram-negative infections. PxB acts by binding to the cell membrane and altering the structure of the membrane, thereby rendering the cell membrane more permeable. Thus, PxB is a cationic, basic protein that acts like a detergent. Although aerosolized PxB has been used in the treatment of cystic fibrosis lung infections, PxB is not generally suitable for parenteral use in humans because it readily self-aggregates in aqueous solution. There is a need in the art to develop new means for the delivery of PxB.

Endotoxins are potentially toxic, natural compounds found inside pathogens such as bacteria and mycobacteria. Classically, an “endotoxin” is a toxin, which unlike an “exotoxin,” is not secreted in soluble form by live bacteria, but is a structural component in the bacteria which is released mainly when bacteria are lysed. The prototypical examples of endotoxin are lipopolysaccharide (LPS) or lipo-oligo-saccharide (LOS) found in the outer membrane of various Gram-negative bacteria. The term “LPS” is often used interchangeably with “endotoxin,” and the term “endotoxin” came from the discovery that portions of Gram-negative bacteria itself can cause toxicity. Studies of endotoxin have revealed that the effects of “endotoxin” are due to LPS. There are, however, endotoxins other than LPS. For example, delta endotoxin of Bacillus thuringiensis makes crystal-like inclusion bodies next to the endospore inside the bacteria, which is toxic to larvae of insects feeding on plants, but is harmless to humans.

Moreover, bacterial endotoxins are present in bacterial vectors used in the production of recombinant proteins, including drugs and vaccines. Such endotoxins can contaminate the recombinantly produced proteins and cause serious adverse effects, including death in animals or humans that receive the recombinantly produced proteins.

Consequently, there is a need in the art to find new ways to deliver water-insoluble antibiotics, like PxB, and there is a need in the art to find new compounds and new ways to treat or even neutralize bacterial endotoxins. The invention provides such new means for the delivery of water-insoluble antibiotics, like PxB, by providing a long-acting, biocompatible and biodegradable parenteral nanoformulation of PxB in the form of sterically stabilized phospholipids nanomicelles. The invention also provides sterically stabilized phospholipids nanomicelles that are effective in neutralizing the effects of endotoxins, exotoxins, and other toxins associated with bacteria, fungi, protozoa, and viruses.

### SUMMARY OF THE INVENTION

The invention provides sterically stabilized phospholipid micellar and liposomal compositions with and without a water-insoluble or amphiphilic antibiotic, antifungal, antiprotozoal, or antiviral agent. The invention further provides methods for the delivery of such compositions in a subject including, but not limited to, a mammalian subject. In one aspect, the mammalian subject is human.

Such compositions provide increased solubility, increased stability, and decreased toxicity or injury. Even further, the invention provides a new use for such sterically stabilized phospholipid micelle and liposome compositions, specifically in decreasing toxicity or injury associated with an exogenous agent. An “exogenous agent” is an agent originating from outside, introduced from outside, or produced outside the organism or system. In one aspect, the exogenous agent is bacterial, mycobacterial, fungal, viral, or protozoal in origin. In another aspect, the sterically stabilized phospholipid micelle or liposome composition optionally comprises one or more antibiotic, antibacterial, antifungal, antiviral, or antiprotozoal agents. In a further aspect, the micelle or liposome composition of the invention comprise a combination of these agents. The invention also includes the use of sterically stabilized phospholipid micelle or liposome compositions in the production and storage of recombinant proteins, wherein the compositions neutralize or decrease toxicity associated with such protein production and storage.

In one embodiment, the invention includes methods of decreasing toxicity or injury associated with an exogenous agent comprising the step of contacting the agent with a sterically stabilized micelle or liposome composition in an amount and under conditions effective to decrease toxicity or injury. Contacting the agent with the micelle or liposome results in a type of binding or capturing the agent in the micelle or liposome, resulting in decreased toxicity or injury of the agent. In one aspect, the micelle or liposome composition may additionally comprise an antibiotic, antibacterial, antifungal, antiviral, antiprotozoal or anti-inflammatory agent. In another aspect, such agent is water-insoluble or hydrophobic or amphiphilic. In a further aspect, the agent is the antibiotic polymyxin B, polymyxin E, or gramicidin. In yet another aspect of the invention, the toxicity is associated with the presence of an endotoxin. In still another aspect, the toxicity is associated with the presence of an exotoxin. In an additional aspect, the toxicity is associated with the presence of an aflatoxin or mycotoxin. In yet another aspect, the toxicity is associated with the presence of a toxin in the viral agent. In still another aspect, the toxicity is associated with the presence of a toxin in the protozoal agent. In another aspect, the sterically stabilized micelle or liposome composition interacts with a hydrophobic domain of the agent, thereby decreasing toxicity or injury caused by the agent.

In another embodiment, the invention includes methods of decreasing toxicity or injury associated with expression of a recombinant peptide, polypeptide, fragment or analog thereof in a host cell transformed or transfected with a polynucleotide encoding the recombinant peptide, polypeptide, fragment or analog thereof comprising the step of contacting a toxin in the culture medium of the host cell with a sterically stabilized micelle or liposome composition before, during, and/or after expression of the recombinant peptide or polypeptide and in an amount and under conditions effective to decrease toxicity or injury. In one aspect, such methods further comprise storing the recombinant peptide, polypeptide, fragment or analog thereof in the presence of a sterically stabilized micelle or liposome composition.

In yet another embodiment, the invention includes methods of decreasing endotoxin-induced or exotoxin-induced activation of a transcription factor in a cell comprising the step of contacting a toxin from the cell with a sterically stabilized micelle or liposome composition. In various aspects, the transcription factor is nuclear factor-kappa B (NF-κB), activator protein-1 (AP-1), or PU.1. In one aspect, the cell is in an inflamed tissue or organ. By attenuating endoxin-induced activation, the micelle or liposome renders the toxin less virulent, i.e., “decreases toxicity.” This method of decreasing toxicity in a cell is useful in the treatment of toxemia, inflammation, infection, bacteremia, sepsis, septic shock, acute lung injury, acute respiratory distress syndrome (ARDS), severe acute respiratory syndrome (SARS), systemic inflammatory response syndrome (SIRS), or multiple organ dysfunction syndrome (MODS). Such methods are also useful in the treatment of tumors including, but not limited to, cancer and cancerous tumors, that are associated with the above-recited conditions.

The terms “decreasing toxicity” and “preventing toxicity” are used herein. In one aspect, it is understood that “decreasing” essentially means “lowering the amount or concentration” of toxicity associated with the toxin or toxic agent, and includes lowering the amount of toxicity to undetectable levels. “Preventing” essentially means “stopping toxicity associated with the toxin or toxic agent before it has a chance to occur.”

The terms “decreasing injury” and “preventing injury” are used herein. In one aspect, it is understood that “decreasing” essentially means “lowering the amount” of injury to a cell or tissue which results from the association of the cell or tissue with the toxin or toxic agent, and includes lowering the amount of injury to undetectable levels. “Preventing” essentially means “stopping injury” associated with the toxin or toxic agent before it has a chance to occur.” Cellular injury appears to be the common denominator in almost all diseases. Injury is an alteration in cell structure or functioning resulting from some stress, including, but not limited to, stress from toxicity, that exceeds the ability of the cell to compensate through normal physiologic adaptive mechanisms. Cellular injury is also brought about disease-producing cellular stresses including, but not limited to, hypoxia, chemical injury, physical agents, infection, immune reactions, nutritional imbalance, genetic derangements, and tumor growth, including, but not limited to, cancer. There is a common pathophysiology between cancer and tissue inflammation and injury, wherein they all comprise leaky vasculature to feed the tissue. Thus, the invention includes treatment of cellular injury or tissue injury associated with cancer.

In a further embodiment, the invention includes sterically stabilized micelle, sterically stabilized mixed micelle, or sterically stabilized liposome compositions comprising a water-insoluble agent, wherein the micelle or liposome configuration prevents aggregate formation of the agent. The invention includes methods of treating an infection in a subject with an effective amount of such compositions. In certain aspects, the infection is caused by one or more types of bacteria, mycobacteria, fungi, virus, or protozoa. In some aspects, the bacteria are Gram-negative. In other aspects, the bacteria are Gram-positive. Thus, in certain aspects, the agent is an antibiotic, antibacterial, antifungal, antiviral, antiprotozoal, antiinflammatory, or immunomodulatory agent. The invention includes all types of water-insoluble antibiotics. In one aspect, the water-insoluble antibiotic is polymyxin B, polymyxin E, or gramicidin. In another aspect, the sterically stable micelle or liposome composition remains stable for at least about 48 hours at about room temperature. In certain aspects, the compositions remains stable for at least about 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 64, 66, 68, 70, and 72 hours. Room temperature normally ranges from about 20° C. to about 26° C. Room temperature therefore includes, but is not limited to temperatures ranging from about 20° C., to about 21° C., to about 22° C., to about 23° C., to about 24° C., to about 25° C., to about 26° C.

In still another embodiment, the invention includes methods of decreasing inflammation or injury in a subject comprising administering to the subject a sterically stabilized micelle or liposome composition in an amount effective to decrease inflammation or injury. In one aspect, the sterically stabilized micelle or liposome composition comprises a water-insoluble or amphiphilic agent. In a further aspect, any water-insoluble or amphiphilic antibiotic is contemplated for use herein. In various aspects, the agent is antibiotic, antibacterial, antifungal, antiviral, antiprotozoal, antiinflammatory, or immunomodulatory. In one aspect, the invention includes methods of treating or preventing a condition associated with toxemia, inflammation, infection, bacteremia, sepsis, septic shock, acute lung injury, acute respiratory distress syndrome (ARDS), severe acute respiratory syndrome (SARS), systemic inflammatory response syndrome (SIRS), or multiple organ dysfunction syndrome (MODS) in a subject comprising the step of administering to the subject the compositions of the invention in an amount effective to treat the condition. In a further aspect, it is contemplated that the compositions are administered to a subject prior to surgery in an amount effective to prevent such conditions.

In another embodiment, the invention includes methods of decreasing inflammation or injury in a subject comprising the step of administering to the subject a sterically stabilized micelle or liposome composition comprising a compound selected from the group consisting of glucagon-like peptide-1 (GLP-1), GLP-2, triggering receptor expressed on myeloid cells (TREM-1) peptide, TREM-2, TREM-3, 17-(allylamino)-17-demethoxygeldanamycin (17-AAG), and fragments and analogs thereof, in an amount and under conditions effective to decrease or eliminate inflammation or injury. Such methods may further comprise administering a combination of one or more compounds selected from the group consisting of GLP-1, GLP-2, TREM-1 peptide, TREM-2, TREM-3, 17-AAG, and fragments and analogs thereof. In some aspects, the compounds are in a D isoform, or an L isoform, or a combination of both D and L isoforms. In various aspects, the compound is linked to the sterically stabilized micelle or liposome composition. In certain aspects, the compound is used to target the micelle or liposome composition to a cell, tissue, or organ. In particular aspects, the inflammation or injury is of the lung or chest.

In yet another embodiment, the invention includes methods of decreasing infection, bacteremia, sepsis, or septic shock in a subject comprising the step of administering to the subject a sterically stabilized micelle or liposome composition comprising vasoactive intestinal peptide (VIP), and fragments and analogs thereof, in an amount and under conditions effective to decrease infection, bacteremia, sepsis, or septic shock. In various aspects, the VIP is in a D isoform, or an L isoform, or a combination of both D and L isoforms. In particular aspects, the infection is ocular.

In still another embodiment, the invention includes methods of treating or preventing hyperglycemia in a subject comprising the step of administering to the subject a sterically stabilized micelle or liposome composition comprising GLP-1, and fragments and analogs thereof, in an amount and under conditions effective to decrease hyperglycemia. In some aspects, the hyperglycemia results from a diabetic condition in the subject. However, the invention is not limited to treating only diabetes as it can be used to treat hyperglycemia resulting from any condition.

The compositions provided may be used for therapeutic or prophylactic purposes by incorporating them with appropriate pharmaceutical carrier materials and administering an effective amount to a subject, such as a human (or other mammal). The invention includes uses of compositions of the invention for the preparation of medicaments. Other related aspects are also provided in the instant invention.

Other features and advantages of the invention will become apparent from the following detailed description. It should be understood, however, that the detailed description and the specific examples, while indicating specific embodiments of the invention, are given by way of illustration only, because various changes and modifications within the spirit and scope of the invention will become apparent to those skilled in the art from this detailed description.

### BRIEF DESCRIPTION OF THE DRAWINGS

FIG. 1 provides results of a normalized luciferase activity of TREM1-luciferase transfected RAW 264.7 cells (mouse macrophages) treated with saline, SSM, sub-micellar concentration of lipid, GLP-SSM (GM) or VIP-SSM (VM) in the presence and absence GLP-1 receptor or VIP receptor antagonists Exendin(9-39) and VIP(6-28), respectively. Inflammation of the macrophages was induced by the addition of Pseudomonas aeruginosa strain PA103 (PA103). * indicates a significant difference from the saline-treated PA103-stimulated group.

FIG. 2 shows a graph depicting the effect of SSM on NF-kappaB activation displayed as relative luminescence units (RLU) normalized to protein concentration in bone marrow-derived macrophages (BMDM) transfected with a NF-kappaB-driven luciferase reporter plasmid. RLU is plotted on the y-axis versus treatments of BMDM on the x-axis.

FIG. 3 shows an intensity weighting particle size distribution (INT-WT NICOMP DISTRIBUTION) of 1 mM DSPE-PEG₂₀₀₀ equilibrated for 2 hours in sterile normal saline; 15-17 nm micelles formed (n=1).

FIG. 4 shows an intensity weighting particle size distribution (INT-WT NICOMP DISTRIBUTION) of 1 mM DSPE-PEG₂₀₀₀ equilibrated for 48 hours in sterile normal saline; 15-17 nm micelles remained stable (n=1).

FIG. 5 shows an intensity weighting particle size distribution (INT-WT NICOMP DISTRIBUTION) of 6.9 mM polymyxin B (PxB) sulfate in sterile normal saline after 2 hours of equilibration at 25° C. (n=2); scale was from 10-10,000 nm and showed no large aggregates.

FIG. 6 shows an intensity weighting particle size distribution (INT-WT NICOMP DISTRIBUTION) of 6.9 mM PxB sulfate in sterile normal saline after 2 hours of equilibration at 25° C. (n=2); scale was from 1-1,000 nm and showed large aggregates at 640 nm.

FIG. 7 shows an intensity weighting particle size distribution (INT-WT NICOMP DISTRIBUTION) of 6.9 mM PxB sulfate in sterile normal saline after 48 hours of equilibration, showing aggregates at 517 nm (n=2)

FIG. 8 shows an intensity weighting particle size distribution (INT-WT NICOMP distribution) of 6.9 mM PxB sulfate with 1 mM DSPE-PEG₂₀₀₀ after 2 hours of equilibration, showing micelles at 15-20 nm and particles smaller than 2 nm (n=2).

FIG. 9 show an intensity weighting particle size distribution (INT-WT NICOMP DISTRIBUTION) of 6.9 mM PxB in sterile normal saline after 48 hours of equilibration showing the presence of a third species of particles at 4 nm in diameter (n=2).

FIG. 10 shows a volume weighting particle size distribution (VOL-WT NICOMP DISTRIBUTION) of 6.9 mM PxB in sterile normal saline after 48 hours of equilibration showing the presence of a third species of particles at 4 nm in diameter (n=2).

FIG. 11 shows an intensity weighting particle size distribution (INT-WT NICOMP DISTRIBUTION) of 2.3 mM PxB in 1 mM DSPE-PEG₂₀₀₀ after 2 hours of equilibration showing the presence of particles at 7 nm in diameter and 17-20 nm in diameter (n=5).

FIG. 12 shows an intensity weighting particle size distribution (INT-WT NICOMP DISTRIBUTION) of 2.3 mM PxB in 1 mM DSPE-PEG₂₀₀₀ after 24 and 48 hours of equilibration showing the decrease of particle size from 7 nm to 2.4 nm in diameter and 17-20 nm in diameter (n=5).

FIG. 13 shows an intensity weighting particle size distribution (INT-WT NICOMP DISTRIBUTION) of 1.0 mM PxB in sterile normal saline after 2 hours of equilibration showing the presence of aggregates occurring at 240 nm in diameter (n=6).

FIG. 14 shows an intensity weighting particle size distribution (INT-WT NICOMP DISTRIBUTION) of 1.0 mM PxB in sterile normal saline after 48 hours of equilibration showing the presence of aggregates occurring at 726 nm in diameter with the presence of smaller aggregates or particles at 45 nm in diameter (n=6).

FIG. 15 shows an intensity weighting particle size distribution (INT-WT NICOMP DISTRIBUTION) of 1.0 mM PxB with 1.0 mM DSPE-PEG₂₀₀₀ after 2 hours of equilibration showing the presence of SSM at 17-20 nm and at 7 nm in diameter (n=7).

FIG. 16 shows an intensity weighting particle size distribution (INT-WT NICOMP DISTRIBUTION) of 1.0 mM PxB with 1.0 mM DSPE-PEG₂₀₀₀ after 48 hours of equilibration showing the presence of SSM at 17-20 nm and particles at 7 nm in diameter (n=7).

FIG. 17 shows an intensity weighting particle size distribution (INT-WT NICOMP DISTRIBUTION) of 0.5 mM PxB in sterile normal saline after 24 and 48 hours of equilibration showing the presence PxB aggregates 520 nm in diameter (n=8).

FIG. 18 shows an intensity weighting particle size distribution (INT-WT NICOMP DISTRIBUTION) of 0.5 mM PxB with 1.0 mM DSPE-PEG₂₀₀₀ after 2 hours of equilibration showing the presence PxB aggregates above 50 nm as well as SSM between 17-20 nm in diameter (n=9).

FIG. 19 shows an intensity weighting particle size distribution (INT-WT NICOMP DISTRIBUTION) of 0.5 mM PxB with 1.0 mM DSPE-PEG₂₀₀₀ after 24 hours of equilibration showing the presence of SSM between 17-20 nm in diameter and residual PxB larger than 20 nm in diameter (n=9).

FIG. 20 shows an intensity weighting particle size distribution (INT-WT NICOMP DISTRIBUTION) of 0.5 mM PxB with 1.0 mM DSPE-PEG₂₀₀₀ after 48 hours of equilibration showing only the presence of SSM between 17-20 nm in diameter (n=9).

### DETAILED DESCRIPTION OF THE INVENTION

The invention provides sterically stabilized phospholipid micellar and liposomal compositions with or without antibiotics. In one aspect, the invention provides phospholipid micellar and liposomal compositions alone for the neutralization of toxins associated with bacterial, mycobacterial, fungal, protozoal, and viral agents. In yet another aspect, the invention provides phospholipid micellar and liposomal compositions comprising antibiotics, antifungal, antiprotozoal, and antiviral agents. The invention also provides methods for the delivery of such compositions in a subject. In on aspect, the subject is a mammal. In a further aspect, the mammal is a human.

The invention also provides phospholipid micellar and liposomal compositions comprising water-insoluble or amphiphilic antibiotics and methods for the delivery of such compositions in a subject. Such compositions provide increased solubility, increased stability, targeted delivery, and decreased toxicity.

In a further aspect, the invention provides sterically stabilized phospholipid micelles or liposomes as novel biocompatible and biodegradable nanocarriers for water-insoluble antibiotics. Such water-insoluble antibiotics include, but are not limited to, polymyxin B (PxB), daptomycin, anthrax toxin, botulism, botox, thiostrepton, ciprofloxacin, rifampicin, gramicidin, amphotericin B, and ketoconazole. Such compositions are particularly useful as an anti-infective drug in the treatment of drug-resistant bacteria.

“Sterically stabilized phospholipid micelles” or “sterically stabilized micelles (SSM)” or “sterically stabilized mixed micelles (SSMM)” or “micelles” or “nanomicelles” are used interchangeably herein. Such terms are known in the art as described, for example, in Ashok et al. (supra) and Rubenstein et al. (Chem. Biol. Interact. 30; 171(2):190-194, 2008) “Sterically stabilized phospholipid liposomes” or “sterically stabilized liposomes (SSL)” or “liposomes” are used interchangeably herein, and are also well known in the art. See, for example (Rubenstein et al., Int. J. Pharm. 316(1-2):144-147, 2006).

Such SSM or SSMM or SSL according to the invention are, in one aspect, produced from one or more lipid materials well known and routinely utilized in the art to produce micelles and liposomes including at least one lipid component covalently bonded to a water-soluble polymer. Such SSM or SSMM or SSL according to the invention are, in one aspect, produced from one or more lipid materials well known and routinely utilized in the art to produce micelles or liposomes and including at least one lipid component covalently bonded to a water-soluble polymer. In various aspects, lipids include relatively rigid varieties, such as sphingomyelin, or fluid types, such as phospholipids having unsaturated acyl chains. The lipid materials are selected by those of skill in the art in order that the circulation time of the micelles or liposomes is balanced with the drug release rate.

Polymers of the invention thus include any compounds known and routinely utilized in the art of sterically stabilized liposome (SSL) technology and technologies which are useful for increasing circulatory half-life for proteins, including for example polyvinyl alcohol, polylactic acid, polyglycolic acid, polyvinylpyrrolidone, polyacrylamide, polyglycerol, polyaxozlines, or synthetic lipids with polymeric head groups. In one aspect, the polymers are water-soluble polymers. Such water soluble polymers include, but are not limited to, polyethylene glycols, copolymers of ethylene glycol/propylene glycol, polyvinyl alcohol, carboxymethylcellulose, polyvinyl pyrrolidone, poly-1,3-dioxolane, poly-1,3,6-trioxane, ethylene/maleic anhydride copolymer, polyaminoacids (either homopolymers or random copolymers), and dextran.

In one aspect, a polymer of the invention is polyethylene glycol” or “PEG”. In a further aspect, “PEG” is a polyalkylene glycol compound or a derivative thereof, with or without coupling agents or derivatization with coupling or activating moieties (e.g., with aldehyde, hydroxysuccinimidyl, hydrazide, thiol, triflate, tresylate, azirdine, oxirane, orthopyridyl disulphide, vinylsulfone, iodoacetamide or a maleimide moiety). “PEG” includes substantially linear, straight chain PEG, branched PEG, or dendritic PEG. (See, e.g., Merrill, U.S. Pat. No. 5,171,264; Harris et al., Multiarmed, monofunctional, polymer for coupling to molecules and surfaces, U.S. Pat. No. 5,932,462; Shen, N-maleimidyl polymer derivatives, U.S. Pat. No. 6,602,498).

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). In the present application, the term “PEG” is used broadly to encompass any polyethylene glycol molecule, in mono-, bi-, or poly-functional form, without regard to size or to modification at an end of the PEG, and can be represented by the formula, X—O(CH₂CH₂O)ₙ—ICH₂CH₂OH, where n is 20 to 2300 and X is H or a terminal modification, e.g., a Q-4 alkyl. In some useful embodiments, a PEG used in the invention terminates on one end with hydroxy or methoxy, i.e., X is H or CH₃ (“methoxy PEG”). It is noted that the other end of the PEG, which is shown in formula above terminating in OH, covalently attaches to an activating moiety via an ether oxygen bond, an amine linkage, or amide linkage. When used in a chemical structure, the term “PEG” includes the formula above without the hydrogen of the hydroxyl group shown, leaving the oxygen available to react with a free carbon atom of a linker to form an ether bond.

Any molecular mass for a PEG can be used as practically desired, e.g., from about 1,000 Daltons (Da) to 100,000 Da (n is 20 to 2300). In one aspect, “PEG” at a molecular weight between 1000 Da and 5000 Da is used in the invention. The number of repeating units “n” in the PEG is approximated for the molecular mass described in Daltons.

In another aspect, lipids for producing micelles or liposomes according to the invention include distearoyl-phosphatidylethanol amine covalently bonded to PEG (PEG-DSPE) alone or in further combination with phosphatidylcholine (PC), and phosphatidylglycerol (PG) in further combination with cholesterol (Chol) and/or calmodulin. In yet a further aspect, lipids for producing micelles or liposomes according to the invention include DSPE-PEG₂₀₀₀.

Although the invention provides working examples using SSM, SSMM and SSL are also contemplated for use herein. Methods of the invention for preparation of SSM or SSMM or SSL compositions are carried out using any of the various techniques known in the art. In one aspect, micelle or liposome components are mixed in an organic solvent and the solvent is removed using either evaporation or lyophilization. Removal of the organic solvent results in a lipid film, or cake, which is subsequently hydrated using an aqueous solution to permit formation of micelles or liposomes. The resulting micelles or liposomes are mixed with an amphiphilic or water-insoluble compound of the invention whereby the amphiphilic or water-insoluble compound associates with the micelle or liposome and assumes a more favorable biologically active conformation.

In another technique, one or more lipids are mixed in an aqueous solution after which the lipids spontaneously form micelles or, with some external energy, form liposomes. The resulting micelles or liposomes are mixed with an amphiphilic or water-insoluble compound which associates with the micelle or liposome products and assumes a more favorable biologically active conformation. Preparing micelle or liposome products by this method is particularly amenable for large scale and safer preparation and requires a considerable shorter time frame than methods previously described. The procedure is inherently safer in that use of organic solvents is eliminated.

The micelles and liposome compositions or products of the invention are characterized by improved stability and biological activity of the compounds which they comprise and are useful in a variety of therapeutic applications. According to one embodiment, the micelles and liposome products can be used for the delivery of biologically active amphiphilic or water-insoluble compounds. In one aspect, the amphiphilic or water-insoluble compound has antibacterial activity. In a further aspect, the amphiphilic or water-insoluble compound is an antibiotic such as, but not limited to, PxB and analogs of PxB. The sterically stable micelle or liposome compositions of the invention are particularly useful in preventing the formation of aggregates of the water-insoluble compounds. In one aspect of the invention, SSM or SSL are useful in preventing aggregates of PxB. Such antibiotic micelle or liposome compositions are useful in a as anti-infective drugs and in the treatment of drug-resistant bacteria. In one aspect, the compound is useful in the treatment of a resistant Gram-negative infection.

The invention includes the use of SSM or SSMM or SSL in decreasing or preventing the effects of bacterial toxigenesis. Toxigenesis, or the ability to produce toxins, is an underlying mechanism by which many bacterial pathogens produce disease. At a chemical level, there are two main types of bacterial toxins, lipopolysaccharides, which are associated with the cell wall of Gram-negative bacteria, and proteins, which are released from bacterial cells and may act at tissue sites removed from the site of bacterial growth. The cell-associated toxins are referred to as endotoxins and the extracellular diffusible toxins are referred to as exotoxins.

Exotoxins are usually secreted by bacteria and act at a site removed from bacterial growth. However, in some cases, exotoxins are only released by lysis of the bacterial cell. Exotoxins are usually proteins, minimally polypeptides, that act enzymatically or through direct action with host cells and stimulate a variety of host responses. Most exotoxins act at tissue sites remote from the original point of bacterial invasion or growth. However, some bacterial exotoxins act at the site of pathogen colonization and may play a role in invasion. Terms such as enterotoxin, neurotoxin, leukocidin or hemolysin are descriptive terms that indicate the target site of some well-defined protein toxins. Bacterial toxins that bring about the death of an animal are known simply as lethal toxins. The invention includes the use of SSM or SSL in decreasing and preventing toxicity associated with all types of exotoxins including, but not limited to, enterotoxin, neurotoxin, leukocidin, and hemolysin.

Endotoxins are cell-associated substances that are structural components of bacteria. Most endotoxins are located in the cell envelope. In one aspect, endotoxin refers to the lipopolysaccharide (LPS) or lipooligosaccharide (LOS) located in the outer membrane of Gram-negative bacteria. Although structural components of cells, soluble endotoxins may be released from growing bacteria or from cells that are lysed as a result of effective host defense mechanisms or by the activities of certain antibiotics. Endotoxins generally act in the vicinity of bacterial growth or presence.

LPS consists of a polysaccharide (sugar) chain and a lipid moiety, known as lipid A, which is responsible for the toxic effects. The polysaccharide chain is highly variable amongst different bacteria. LPS, which is found in the circulation during sepsis, induces cytokine release, hypotension, and death. LPS also induces the metabolic responses seen during infection. The term “lipopolysaccharide” or “LPS” is often used exchangeably with “endotoxin”, owing to its historical discovery. The term “endotoxin” came from the discovery that portions of Gram-negative bacteria itself can cause toxicity, hence the name endotoxin. Studies of endotoxin revealed that the effects of “endotoxin” were in fact due to LPS. There are, however, endotoxins other than LPS. Lipoteichoic acid (LTA), a heat-stable component of the cell membrane and wall of most Gram-positive bacteria, has structural and functional similarities to LPS. Furthermore, LTA induces circulatory shock and treatment of macrophages or adherent mononuclear cells with LTA has been shown to induce cytokine mediators of septic shock (Bhakdi et al., Infect. Immun. 59:4614-4620, 1991). The invention includes the use of SSM or SSL in decreasing or preventing the toxicity associated with all types of endotoxins including, but not limited to, LPS and LTA.

Endotoxins are approximately 10 kDa in size but can form large aggregates up to 1000 kDa. Humans are able to produce antibodies to endotoxins after exposure, but these antibodies are generally directed at the polysaccharide chain and do not protect against a wide variety of endotoxins. Injection of a small amount of endotoxin in human volunteers produced fever, a lowering of the blood pressure, and activation of inflammation and coagulation. Endotoxins are in large part responsible for the dramatic clinical manifestations of infections with pathogenic Gram-negative bacteria, such as Neisseria meningitidis, the pathogen that causes fulminant meningitis.

The invention includes the use of SSM or SSMM or SSL in decreasing the toxicity or injury of all types of bacterial toxins associated with the production and storage of recombinant proteins. It is known that endotoxins, exotoxins, and bacterial enzymes can cause serious adverse events or even death in a mammal. Lipids, such as SSM or SSMM or SSL, can neutralize the effects of endotoxins, exotoxins, and bacterial enzymes by their association with these toxins during the recombination process and/or storage of the protein(s), thereby circumventing interactions of endotoxins, exotoxins, and bacterial enzymes with target cells and minimizing damage from these bacterial toxins.

The invention includes the use of SSM or SSMM or SSL in decreasing or preventing the effects of fungal mycotoxins and aflatoxins. A mycotoxin is a toxic secondary metabolite produced by an organism of the fungus kingdom, including mushrooms, molds, and yeasts. Toxigenesis, or the ability to produce toxins, is an underlying mechanism by which many mycotoxins produce disease. The production of toxins depends on the surrounding intrinsic and extrinsic environments and the toxins vary greatly in their severity, depending on the organism infected and its susceptibility, metabolism, and defense mechanisms. Some of the health effects found in animals and humans include death, identifiable diseases or health problems, and weakened immune systems.

The invention includes the use of SSM or SSMM or SSL in decreasing or preventing the cellular injury, toxicity or damage associated with viruses. Viruses have the ability to produce temporary or permanent damage in a host via cell lysis, production of toxic substances, cell transformation, production of cellular products not normally produced by the cell, and induction of structural alterations in a host cell. Some viruses enter host cells or tissues directly by trauma or insect bite, but most infections start on the mucous membranes of the respiratory and alimentary tracts.

The invention includes the use of SSM or SSMM or SSL in decreasing or preventing the cellular injury, toxicity or damage associated with protozoan. Protozoa are single-celled organisms. The Trichimonas vaginalis organism feeds on bacteria and white blood cells and can live outside the body. The Trypanosoma organism lives in the blood, lymph nodes, spleen, and cerebrospinal fluid of the vertebrate host. The trypanosomes do not actually invade or live in cells. Instead, they inhabit spaces in connective tissue in various organs.

The invention includes the use of SSM or SSMM or SSL with one or more biologically active compound(s) and one or more targeting compound(s). In certain aspects, the targeting compound(s) associates with said SSM, SSMM, or SSL. In one aspect, the targeting compound is linked to one or more lipid components of the micelle. In various aspects, linkage between the targeting compound and the lipid is effected by covalent means in a manner that permits the targeting compound to interact with its cognate receptor, ligand, or binding partner and position the SSM, SSMM, or SSL in close proximity. U.S. Publication Nos. 20020114829, 20020115609, and 20050025819 are each incorporated herein by reference in their entireties. These publication provide additional information relating to SSM, SSMM or SSL and targeting of said SSM, SSMM, or SSL.

The invention includes the use of SSM or SSMM or SSL for decreasing the expression of transcription factors that are involved in the inflammatory response. Such transcription factors include, but are not limited to, the pro-inflammatory transcription factors activator protein-1 (AP-1), nuclear factor-kappa B (NF-κB), and PU.1.

The compositions of the invention are, in one aspect, used to prevent or to treat any of a large number of diseases and conditions associated with endotoxemia, sepsis, or septic shock. In one embodiment, the compositions and methods of the invention are used in conjunction with any type of surgery or medical procedure, when appropriate, that could lead to the occurrence of endotoxemia or related complications (e.g., sepsis syndrome). As a specific example, the invention is used in conjunction with cardiac surgery (e.g., coronary artery bypass graft, cardiopulmonary bypass, and/or valve replacement), transplantation (of, e.g., liver, heart, kidney, or bone marrow), cancer surgery (e.g., removal of a tumor), or any abdominal surgery (see, e.g., PCT/US01/01273).

Additional examples of surgical procedures with which the compositions and methods of the invention are used, when appropriate, include without limitation surgery for treating acute pancreatitis, inflammatory bowel disease, placement of a transjugular intrahepatic portosystemic stent shunt, hepatic resection, burn wound revision, and burn wound escharectomy.

The compositions of the invention are also used in conjunction with non-surgical procedures in which the gastrointestinal tract is compromised. For example, the compositions are used in association with chemotherapy or radiation therapy in the treatment of cancer. The compositions and methods of the invention are also used in the treatment of conditions associated with HIV infection, trauma, or respiratory distress syndrome, as well as with immunological disorders, such as graft-versus-host disease or allograft rejection. Pulmonary bacterial infection and pulmonary symptomatic exposure to endotoxin is also treated using the compositions and methods of the invention (see, e.g., PCT/US00/02173).

The compositions of the invention are also used in the treatment of inflammation. Such compositions are used in the treatment of both acute and chronic inflammation. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterized by simultaneous destruction and healing of the tissue from the inflammatory process. The inflammation may be caused by without limitation burns, chemical irritants, frostbite, toxins, infection by pathogens, physical injury, immune reactions, ionizing radiation, or foreign bodies, such as splinters or dirt.

The compositions of the invention are also used in the treatment of infection including, in various aspects, sepsis. Both inflammation and infection are included in the methods of the invention because the host's response to infection is inflammation. The infection can be bacterial, viral, tubercular, or fungal.

The bioactive compounds in nanomicelles or liposomes are used alone or in combination with other agents in the treatment of eye disorders such as, but not limited to, infection (e.g. bacterial, viral, parasitic, and the like), inflammation (e.g. conjunctivitis, keratitis, uveitis, retinitis, and the like), allergy, dry eye, Sjogren's Syndrome, and glaucoma.

The invention further provides methods of administering a biologically active amphiphilic or water-insoluble compound to a mammal to treat a generalized infection or to a target tissue comprising the steps of: preparing a biologically active micelle or liposome product comprising a biologically active amphiphilic compound in association with a micelle or liposome product according to the methods of the invention and administering a therapeutically effective amount of the micelle or liposome product to the target tissue. The micelle or liposome products of the invention are in various aspects administered intravenously, intraarterially, intranasally, such as by aerosol administration, nebulization, inhalation, or insufflation, intratracheally, intraarticularly, orally, sublingually, transdermally, subcutaneously, vaginally, intrarectally, topically onto mucous membranes, such as, but not limited to, oral mucosa, lower gastrointestinal mucosa and conjunctiva, and directly onto target tissues. Methods of administration for amphiphilic compounds are equally amenable to administration of compounds that are insoluble in aqueous solutions.

Biologically active compounds, such as water-insoluble antibiotics, are administered at significantly reduced dosage levels as compared to administration of the compound alone, particularly wherein the compound has a particularly short half life or lowered bioactivity in circulation. For example PxB (Bedford Labs, Bedford, Ohio) is approved by the FDA for the treatment of acute infections of the urinary tract, meninges and bloodstream caused by Gram-negative bacteria. It has been approved for parenteral, intramuscular, intrathecal, intravenous, and ophthalmic administration. The PxB-SSM composition of the invention are contemplated for delivery at lower dosages than currently approved by the FDA due to their increased stability, increased solubility, and decreased toxicity.

Regardless of which bioactive compound is associated with the SSM or SSMM or SSL, the micelle or liposome product is in one aspect tested in order to determine a biologically effective amount required to achieve the same result effected by the compound administered by conventional means. The worker of ordinary skill in the art would realize that the biologically effective amount of a particular compound when delivered by conventional means would serve as a starting point in the determination of an effective amount of the compound in SSM or SSMM or SSL. It would therefore be highly predictive that the same and lesser dosages of the same compound in SSM or SSMM or SSL would be effective as well and merely routine to determine the minimum dosage required to achieve a desired biological effect.

In certain aspects, a bioactive compound of the invention is glucagon-like peptide-1 (GLP-1), and biologically active fragments and analogs thereof. GLP-1 increases insulin secretion from the pancreas in a glucose-dependent manner, decreases glucagon secretion from the pancreas, increases beta cells mass and insulin gene expression, inhibits acid secretion and gastric emptying in the stomach, decreases food intake by increasing satiety, promotes insulin sensitivity, and exhibits anti-inflammatory effects. Thus, GLP-1 possesses several physiological properties that make it (and biologically active fragments and analogs) useful in the treatment of hyperglycemia and diabetes mellitus. GLP-1(7-36) is a 30-amino acid incretin hormone that has been shown to exhibit glucose lowering and anti-inflammatory properties (Iwai et al., Neurosci. Res. 55: 352, 2006; Baggio et al., Gastroenterol. 132: 2131, 2007). The biologically active forms of GLP-1 are GLP-1(7-37) and GLP-1(7-36). In various aspects, either form of biologically active GLP-1 or a biologically active fragment or analog thereof is used in the invention, and the terms “GLP-1,” “GLP-1(7-37),” and “GLP-1(7-36)” are used interchangeably herein. GLP-1(7-36) is a 30-amino acid incretin hormone that has been shown to exhibit glucose lowering and anti-inflammatory properties (Iwai et al., Neurosci. Res. 55: 352, 2006; Baggio et al., Gasiroenterol. 132: 2131, 2007). However, the clinical application of GLP-1 has been hampered by a short plasma half-life due to rapid enzymatic degradation and renal clearance. Therefore, by loading GLP-1 or a biologically active fragment or analog thereof into SSM, SSMM, or SSL, the half-life of GLP-1 increases both in vitro and in vivo. GLP-1 then can be stored for longer periods of time and can stay in the body for a longer period of time to elicit biological activity. GLP-1 in saline is in a random coil or unstructured state leaving it vulnerable to enzymatic degradation; whereas, GLP-1 in SSM is in an alpha-helical structure and thus protected from degradation (Sreerama et al., Biochemistry 33:10022-25, 1994). The invention includes the use of GLP-1 in SSM, SSMM, or SSL in the treatment of infection or inflammation, and in the treatment of diabetes, hyperglycemia, and related disorders.

In an additional aspect, glucagon-like peptide-2 (GLP-2) and biologically active fragments and analogs thereof, is another such bioactive compound that is used in the invention. GLP-2 is used alone or in combination with other peptides, fragments or analogs thereof as described herein. Human GLP-2 is a 33-amino acid peptide. GLP-2 is created by specific post-translational proteolytic cleavage of proglucagon in a process that also liberates the related glucagon-like peptide-1 (GLP-1). GLP-2 is produced by the intestinal endocrine L cell and by various neurons in the central nervous system. Intestinal GLP-2 is co-secreted along with GLP-1 upon nutrient ingestion. When externally administered, GLP-2 produces a number of effects, including intestinal growth, enhancement of intestinal function, reduction in bone breakdown and neuroprotection. GLP-2 may act in an endocrine fashion to link intestinal growth and metabolism with nutrient intake. GLP-2 and related analogs are used for treatment of short bowel syndrome, Crohn's disease, necrotizing enterocolitis, osteoporosis and as adjuvant therapy during cancer chemotherapy.

In other aspects of the invention, vasoactive intestinal peptide (VIP), and biologically active fragments and analogs thereof, is another such bioactive compound that is used in the invention. VIP as discussed in U.S. Pat. No. 6,322,810 is hereby incorporated by reference in its entirety. VIP or a biologically active fragment or analog thereof is loaded into SSM, SSMM, or SSL for achieving an improved biological effect. VIP is a peptide hormone containing 28 amino acid residues and is produced in many areas of the human body including the gut, pancreas, and suprachiasmatic nuclei of the hypothalamus in the brain. VIP has many different effects on various parts of the body and is shown herein to be useful in the treatment of infection. In the digestive system, VIP induces smooth muscle relaxation (lower esophageal sphincter, stomach, and gallbladder), stimulates secretion of water into pancreatic juice and bile, and causes inhibition of gastric acid secretion and absorption from the intestinal lumen. Its role in the intestine is to greatly stimulate secretion of water and electrolytes, as well as dilating intestinal smooth muscle, dilating peripheral blood vessels, stimulating pancreatic bicarbonate secretion, and inhibiting gastrin-stimulated gastric acid secretion. These effects work together to increase motility. In the brain, VIP is involved in synchronizing the timing of suprachiasmatic nucleus function with the environmental light-dark cycle, making VIP a crucial component of the mammalian circadian timekeeping machinery. VIP also functions in regulating prolactin secretion and stimulating prolactin release. VIP is also found in the heart and has significant effects on the cardiovascular system. VIP has a short half-life in the blood, and its half life is increased by loading it a micelle or liposome. The invention includes the use of VIP in SSM, SSMM, or SSL in the treatment of infection, inflammation, and related disorders.

In a further aspect, the invention includes the use of GLP-1, GLP-2, or VIP in SSM, SSMM, or SSL in the treatment of conditions including, but not limited to, chemotherapy-induced gastrointestinal mucositis, necrotizing enterocolitis, short bowel syndrome, inflammatory bowel disease, food allergy, monoelusive mesenteric ischemia or gut ischemia, portal hypertension, and ischemic colitis.

In another aspect of the invention, 17-allylamino-17-demethoxygeldanamycin (17-AAG), and biologically active fragments and analogs thereof, is a bioactive compound that is used in the invention. 17-AAG or a biologically active fragment or analog thereof is loaded into SSM, SSMM, or SSL to achieve an improved biological effect in vitro or in vivo. 17-AAG, a potent heat shock protein 90 (Hsp90) inhibitor, belongs to a family of a benzoquinone ansamycins, which includes geldanamycin and derivates thereof, such as 17-DMAG. Geldanamycin induces the degradation of proteins that are mutated in tumor cells, such as v-src, bcr-abl and p53, preferentially over their normal cellular counterparts via Hsp90. The invention includes the use of 17-AAG or one of its analogs in SSM, SSMM, or SSL in the treatment of infection, inflammation, and related disorders.

In a further aspect of the invention, triggering receptor expressed on myeloid cells (TREM-1) peptide, also known as LP17 or TREM-1 binding protein (T1BP), is a bioactive compound for use in the invention. LP17, a 17-amino acid peptide (LQVTDSGLYRCVIYHPP (SEQ ID NO: 1)), is loaded into SSM, SSMM, or SSL to achieve an improved biological effect in vivo. LP17 is a synthetic soluble TREM-1 decoy receptor which functions as a TREM-1 inhibitor. Because TREM-1 has been shown to induce the expression of pro-inflammatory cytokines, TREM-1 is a target for the treatment of chronic inflammatory disorders, including inflammatory bowel disease, and in the treatment of infection including, in various aspects, sepsis. Blocking TREM-1 by the administration of an antagonistic peptide, such as LP17, is one means of treating such diseases and disorders.

In yet another aspect of the invention, TREM-2 or TREM-3, and biologically active fragments and analogs thereof, is a bioactive compound that is used in the invention. Unlike TREM-1, TREM-2 and TREM-3 function to reduce the inflammatory response, not induce inflammatory cytokines. Thus, TREM-2 or TREM-3, and fragments and analogs thereof (and not inhibitors of said proteins, like L17) is also a target for the treatment of chronic inflammatory disorders, including inflammatory bowel disease, and in the treatment of infection including, in various aspects, sepsis. Thus, the delivery of TREM-2 or TREM-3 in SSM, SSMM, or SSL is another means of treating such diseases and disorders.

The invention includes both “L” and “D” stereoisomers (L- and D-isomers, or L- and D-isoforms) of the bioactive compounds discussed herein, and fragments and derivatives thereof. D-isomers act as receptor antagonists in tissues expressing their respective ligands and can be used for treatment, imaging, and active targeting of the nanoformulations of the invention. A D-amino acid peptide inhibitor of NF-κB nuclear localization has been shown efficacious in models of inflammatory disease (Fujihara et al., J. Immunol., 165: 1004-1012, 2000). Other peptide inhibitors have been shown to contain predominantly D-amino acids (see U.S. Pat. No. 5,753,628). The L- and D-isomers of the bioactive compounds are targeted to inflamed and injured cells, tissues and organs. In various aspects, the injured cells, tissues, and organs are tumorous. In certain aspects, the injured cells, tissues, and organs are cancerous. The invention also includes combinations of L-isoforms with D-isoforms. In another aspect, the invention includes bioactive compounds comprising non-naturally occurring amino acid derivatives.

In one aspect, the association of a biologically active amphiphilic or water-insoluble compound with SSM, SSMM, or SSL product, respectively, of the invention increases the magnitude of the biological effects of the compound from about 50 to 100% over the effects observed following administration of the compound alone. Likewise, in another aspect, the association with SSM, SSMM or SSL of the invention invokes a longer lasting biological effect.

The therapeutic methods of the invention include methods for the amelioration of disorders associated with inflammation, infection and antibiotic-resistance and the treatment or neutralization of endotoxins. “Inflammation” as used herein refers to a localized, protective response elicited by injury or destruction of tissues, which serves to destroy, dilute or wall off (sequester) both the injurious agent and the injured tissue. Inflammation is notably associated with influx of leukocytes and or neutrophil chemotaxis. Inflammation may result from infection with pathogenic organisms and viruses and from noninfectious means such as trauma or reperfusion following myocardial infarction or stroke, immune response to foreign antigen, and autoimmune responses. Accordingly, inflammatory disorders amenable to the invention encompass disorders associated with reactions of the specific defense system as well as with reactions of the non-specific defense system.

Therapeutic compositions are also included in the invention. Such compositions comprise a therapeutically effective amount of a micelle or liposome composition alone or in admixture with a pharmaceutically or physiologically acceptable formulation agent selected for suitability with the mode of administration. Such therapeutic compositions include, but are not limited to, micelle or liposome compositions alone. In addition, such therapeutic compositions may also include, but are not limited to, the bioactive compounds discussed herein above. Pharmaceutical compositions comprise a therapeutically effective amount of one or more micelle or liposome compositions in admixture with a pharmaceutically or physiologically acceptable formulation agent selected for suitability with the mode of administration. If a bioactive compound is added to the micelle or liposome compositions, a therapeutically effective amount of such compound is also used.

The therapeutic methods and compositions of the invention are also employed, alone or in combination with other bioactive agents in the treatment of diseases or disorders discussed herein. These preparations of the invention are useful in treating some forms of inflammation, infection, diabetes, hyperglycemia, and other related disorders.

The pharmaceutical composition contain in various aspects formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen sulfite); buffers (such as borate, bicarbonate, Tris HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta cyclodextrin or hydroxypropyl beta cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (in one aspect, sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

The route of administration of the pharmaceutical composition is in accord with known methods. The composition in one aspect is delivered orally. In other aspects the composition is delivered parenterally through injection by intravenous, intraperitoneal, intracerebral (intraparenchymal), intracerebroventricular, intracerebrospinal, intramuscular, intraocular, intraarterial, intraarticular, intraportal, intrarectal, intranasal, or intralesional routes. In addition, a composition of the invention can be introduced for treatment into a mammal by other modes, such as but not limited to, intratumor, topical, subconjunctival, intrabladder, intravaginal, epidural, intracostal, intradermal, inhalation, transdermal, transserosal, intrabuccal, dissolution in the mouth or other body cavities, instillation to the airway, insuflation through the airway, injection into vessels, tumors, organ and the like, and injection or deposition into cavities in the body of a mammal.

Where desired, the composition is administered by bolus injection or continuously by infusion, or by implantation device. Alternatively or additionally, the composition is administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed release bolus, or continuous administration.

In some cases, it may be desirable to use compositions in an ex vivo manner. In such instances, cells, tissues, or organs that have been removed from the patient are exposed to compositions after which the cells, tissues and/or organs are subsequently implanted back into the patient.

In one embodiment, a pharmaceutical composition is formulated for inhalation. For example and without limitation, a micelle or liposome composition is formulated as a dry powder for inhalation. Alternatively, a pharmaceutical micelle or liposome composition inhalation solution is also formulated with a propellant for aerosol delivery. In yet another embodiment, the solution is nebulized. Pulmonary administration is further described in PCT Application No. PCT/US94/001875, which describes pulmonary delivery of chemically modified proteins.

In another embodiment, a pharmaceutical composition is formulated for oral delivery. A micelle or liposome composition which is administered in this fashion is formulated with or without those carriers customarily used in the compounding of solid dosage forms such as tablets and capsules. For example, in one aspect a capsule is designed to release the active portion of the formulation at the point in the gastrointestinal tract when bioavailability is maximized and pre systemic degradation is minimized. Additional agents are optionally included to facilitate absorption of the micelle or liposome composition, including for example and without limitation, diluents, flavorings, low melting point waxes, vegetable oils, lubricants, suspending agents, tablet disintegrating agents, and binders.

Another micelle or liposome composition of the invention comprises an effective quantity of micelle or liposome compositions in a mixture with nontoxic excipients which are suitable for the manufacture of tablets and/or capsules. By dissolving the tablets or capsules in sterile water, or other appropriate vehicle, solutions are prepared in one aspect in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional micelle or liposome compositions will be evident to those skilled in the art, including formulations involving micelle or lipid compositions in sustained or controlled delivery formulations. Techniques for formulating a variety of other sustained or controlled delivery means, such as liposome carriers, bio erodible microparticles or porous beads and depot injections, are also known to those skilled in the art.

Once the composition has been formulated, it may be stored in sterile vials as a solution, suspension, gel, emulsion, solid, or a dehydrated or lyophilized powder. Sterility is achieved in one aspect by filtration through sterile filtration membranes. Where the composition is lyophilized, sterilization using this method may be conducted either prior to or following lyophilization and reconstitution. In addition, parenteral compositions generally are placed into a container having a sterile access port, for example, an intravenous solution bag or vial having a stopper pierceable by a hypodermic injection needle. Such formulations are stored either in a ready to use form or in a form (e.g., lyophilized) requiring reconstitution prior to administration.

A “therapeutically effective dose,” “effective dose,” or “effective amount” of a bioactive compound, or micelle or liposome composition, refers to that amount of the compound sufficient to result in amelioration of one or more symptoms of the disease or disorder being treated. When applied to an individual active ingredient, administered alone, a therapeutically effective amount refers to that ingredient alone. When applied to a combination, a therapeutically effective amount refers to combined amounts of the active ingredients that result in the therapeutic effect, whether administered serially or simultaneously. The invention specifically contemplates that one or more bioactive compounds, or combination of bioactive compounds, and one or more micelle or liposome compositions, may be administered according to methods of the invention, each in an effective amount. Thus, the invention includes the use of a combination of any two, three, four, or more peptides or antibiotics selected from the group consisting of GLP-1, LP-17, VIP, 17-AAG, polymyxin B, polymyxin E, gramicidin, and biologically active fragments and analogs thereof, to treat inflammation, infection, or a related disorder. The invention also includes combinations of micelle or liposome compositions in the treatment of inflammation, infection, or a related disorder.

An effective amount of a composition to be employed therapeutically will depend, for example, upon the therapeutic context and objectives. One skilled in the art will appreciate that the appropriate dosage levels for treatment will thus vary depending, in part, upon the molecule delivered, the indication for which the micelle or liposome composition is being used, the route of administration, and the size (body weight, body surface or organ size) and condition (the age and general health) of the patient. Accordingly, the clinician may titer the dosage and modify the route of administration to obtain the optimal therapeutic effect.

An exemplary regimen would include administration of from 0.001 mg/kg body weight to about 1000 mg/kg, from about 0.01 mg/kg to about 100 mg/kg, from about 0.1 mg/kg to about 100 mg/kg, about 1.0 mg/kg to about 50 mg/kg, or from about 1 mg/kg to about 20 mg/kg, given in daily doses or in equivalent doses at longer or shorter intervals, e.g., every other day, twice weekly, weekly, monthly, semi-annually, or even twice or three times daily. Alternatively, dosages may be measured in international units (IU) ranging from about 0.001 IU/kg body weight to about 1000 IU/kg, from about 0.01 IU/kg to about 100 IU/kg, from about 0.1 IU/kg to about 100 IU/kg, from about 1 IU/kg to about 100 IU/kg, from about 1 IU/kg to about 50 IU/kg, or from about 1 IU/kg to about 20 IU/kg. Administration may be oral, intravenous, subcutaneous, intranasal, inhalation, transdermal, transmucosal, or by any other route discussed herein.

The frequency of dosing will depend upon the pharmacokinetic parameters of the micelle or liposome composition in the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via implantation device or catheter. Further refinement of the appropriate dosage is routinely made by those of ordinary skill in the art and is within the ambit of tasks routinely performed by them. Appropriate dosages may be ascertained through use of appropriate dose response data.

A single bolus injection may be given by intravenous infusion through, for example, a central access line or a peripheral venous line, or by direct injection, using a syringe. Such administration may be desirable if a patient is only at short-term risk for exposure to endotoxin, and thus does not need prolonged persistence of the drug. For example, this mode of administration may be desirable in surgical patients, if appropriate, such as patients having cardiac surgery, e.g., coronary artery bypass graft surgery and/or valve replacement surgery. In these patients, a single bolus infusion of drug can be administered over a period of four hours prior to and/or during surgery. (Note that the amount of drug administered is based on the weight and condition of the patient and is determined by the skilled practitioner.) Shorter or longer time periods of administration can be used, as determined to be appropriate by one of skill in this art.

In cases in which longer-term delivery of a compound of the invention is desirable, for example, in the treatment of a condition associated with long-term exposure to endotoxin, such as during infection or sepsis, or in appropriate surgical situations in which it is determined that prolonged treatment is desirable, intermittent administration can be carried out. In these methods, a loading dose is administered, followed by either (i) a second loading dose and a maintenance dose (or doses), or (ii) a maintenance dose or doses, without a second loading dose, as determined to be appropriate by one of skill in this art.

To achieve further delivery of the compound in a patient, a maintenance dose (or doses) of the compound can be administered, so that levels of the compound are maintained in the blood of a patient. Maintenance doses can be administered at levels that are less than the loading dose(s), for example, at a level that is about ⅙ of the loading dose. Specific amounts to be administered in maintenance doses can be determined by a medical professional, with the goal that the compound level is at least maintained. Maintenance doses can be administered, for example, for about 2 hours every 12 hours beginning at hour 24 and continuing at, for example, hours 36, 48, 60, 72, 84, 96, 108, and 120. Of course, maintenance doses can be stopped at any point during this time frame, as determined to be appropriate by a medical professional.

The infusion methods described above can be carried out using catheters (e.g., peripheral venous, central venous, or pulmonary artery catheters) and related products (e.g., infusion pumps and tubing) that are widely available in the art. One criterion that is important to consider in selecting a catheter and/or tubing to use in these methods is the impact of the material of these products (or coatings on these products) on the micelle or liposome size of the drug.

Additional catheter-related products that can be used in the methods of the invention can be identified by determining whether the material of the products alters micelle or liposome size of the compound, under conditions consistent with those that are used in drug administration. In addition, in the event that a patient already has a catheter in place that does not maintain optimal drug micelle or liposome size, a catheter insert that is made of a compatible material (e.g., a polyamide polymer) or that includes a compatible coating can be used so that the drug solution does not contact the surface of the incompatible catheter. Such an insert, having an outside diameter that is small enough to enable it to be easily inserted into the existing catheter, while maintaining an inside diameter that is large enough to accommodate solution flow of the compound, is placed within the existing catheter and connected to tubing or a syringe through which the drug is delivered.

In the case of pulmonary bacterial infection or pulmonary symptomatic exposure to endotoxin, administration of the compositions of the invention can be effected by means of periodic bolus administration, by continuous, metered inhalation, or by a combination of the two. A single dose may be administered by inhalation as well. Of course, recalcitrant disease may require administration of relatively high doses, the appropriate amounts of which can be determined by one of skill in the art. Appropriate frequency of administration can be determined by one of skill in the art and can be administered several times per day. The compositions of the invention may also be administered once each day or once every other day. In the case of acute administration, treatment is typically carried out for periods of hours or days, while chronic treatment can be carried out for weeks, months, or even years.

Both chronic and acute administration can employ standard pulmonary drug administration formulations, which can be made from the formulations described elsewhere herein. Administration by this route offers several advantages, for example, rapid onset of action by administering the drug to the desired site of action, at higher local concentrations. Pulmonary drug formulations are generally categorized as nebulized (see, e.g., Flament et al., Drug Development and Industrial Pharmacy 21(20):2263-2285, 1995) and aerosolized (Sciarra, “Aerosols,” Chapter 92 in Remington's Pharmaceutical Sciences, 16th edition (ed. A. Osol), pp. 1614-1628; Malcolmson et al., PSTT 1(9):394-398, 1998, and Newman et al., “Development of New Inhalers for Aerosol Therapy,” in Proceedings of the Second International Conference on the Pharmaceutical Aerosol, pp. 1-20) formulations.

### EXAMPLES

The invention is described in more detail with reference to the following non-limiting examples, which are offered to more fully illustrate the invention, but are not to be construed as limiting the scope thereof. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention. All patents and publications mentioned herein are incorporated by reference.

### Example 1

### Sterically Stabilized Micelles Suppress the Bacterial-Induced Inflammatory Response on Macrophages

It was previously determined that when murine macrophages were treated with SSM, the resulting inflammatory response induced by Pseudomonas aeruginosa (P. aeruginosa strain PA103) was significantly lower than the inflammatory response in saline-treated cells. The same anti-inflammatory effect was observed for murine macrophages (RAW 246.7 cells) treated with GLP-SSM or VIP-SSM. GLP-1 and VIP were used in the experiments because they act as immunomodulators in that they suppress excessive inflammation and abnormal immune responses while at the same time promote cell and tissue repair mechanisms (Hahm et al., J. Endocrinol. Invest. 31: 334-340, 2008; Iwai et al., Neurosci. Res. 55: 352-360, 2006). VIP is an endogenous anti-inflammatory mediator, which has been speculated to extend the range of therapeutic treatments available for various disorders, including acute and chronic inflammatory diseases, septic shock and autoimmune diseases (Pozo, Trends in Molecular Medicine 9: 211-217, 2003).

To determine if the anti-inflammatory activity of SSM against P. aeruginosa was due at least in part to phospholipids monomers, the experiment was repeated with RAW 246.7 cells treated with sub-micellular concentration of DSPE-PEG₂₀₀₀ (<1 μM). In addition, the receptor specificity of the anti-inflammatory activities of SSM on macrophages, GLP-SSM and VIP-SSM anti-inflammatory effects were determined by the addition of the GLP-1 receptor antagonist (Exendin(9-39)) (Bregenholt et al., Biochem. Biophys. Res. Commun. 330: 577-584, 2005) and the VIP receptor antagonist (VIP(6-28)) (Mohney et al., J. Neurosci. 18:5285-5293, 1998), respectively.

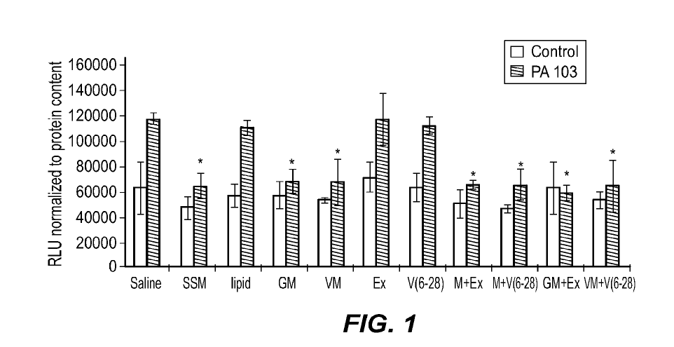

Murine macrophages (RAW 264.7 cells) were transfected with a pro-inflammatory mediator ‘triggering receptor expressed on myeloid cells’ (TREM-1) promoter-driven luciferase gene. TREM-1 is an immunoglobulin superfamily (IgSF) molecule that amplifies inflammation and is a crucial mediator of septic shock (Bouchon et al., Nature 410: 1103-1107, 2001). The macrophages were pre-treated for 18 hr with the following treatments: (i) saline, (ii) SSM, (iii) sub-micellar concentration of lipid (lipid), (iv) GLP-SSM (GM), or (v) VIP-SSM (VM), in the presence or absence of Exendin(9-39) or (VIP(6-28). To allow for the presence of excess antagonist to compete with its respective peptide agonist for receptor binding, the concentration of each receptor antagonist used (10 μM) was 10 times greater than its peptide agonist (1 μM). Inflammation of cells was induced by the addition of the Gram-negative bacteria Pseudomonas aeruginosa (P. aeruginosa strain PA103) for an additional 24 hr, and luciferase activities were measured (see FIG. 1).

The following materials were used in the experiments: TREM1-luciferase RAW 246.7 cells [Source: Mice, TREM1-driven luciferase reporter construct]; P. aeruginosa strain PA103 (American Type Culture Collection (ATCC), Manassas, Va.) glucagon-like peptide I (7-36) (MW 3297.5, Cat#46-1-13B, American Peptide); vasoactive intestinal peptide (MW 3325.9, RRC synthesized peptide); Exendin(9-39) (MW 3369.8, Cat#46-3-10B, American Peptide); VIP(6-28) (MW 2816.32, Cat#H-2066, Bachem); DSPE-PEG₂₀₀₀ (MW 2810, Cat#: PE 18:0/18:0-PEG 2000, Lot#899346-1/09, Lipoid); phosphate-buffere saline (PBS, Cellgro); DMEM cell culture medium (Cellgro) containing 10% fetal calf serum (FCS) (Hyclone), penicillin (100 U/ml)/streptomycin (100 μg/ml) (Invitrogen); DMEM with no phenol red (Cat#21063); and luciferase assay kits (Cat#1500, Promega).

In addition, the following materials were used in different aspect of the invention: Lipids: L-α-egg yolk phosphatidylcholine type V-E in chloroform:methanol (9:1) (Lot #34H8395, and 75H8368), L-α-egg yolk phosphatidyl-D-α-Glycerol in chloroform:methanol (98:2) (Lot #72H8431, and 85H8395), and cholesterol (Lot #60H0476) from Sigma Chemical Co. (St. Louis, Mo.). Di-Palmitoyl-phosphatidyl choline (Lot #LP-04-01-112-187) from Sygenal Ltd. (Switzerland). PEG-DSPE in lyophilized powder form (Lot #180PHG2PK-26) from Avanti Polar Lipids Inc. (Alabaster, Ala.). Various chemicals: trehalose (Lot #43H7060), 2,4-diaminophenol (amidol, Lot #74H3652), ammonium molybdate (Lot #42H3506), sodium bisulfite (Lot #41H09432), HEPES (Lot #43H5720), and sodium chloride (Lot #22H0724) from Sigma Chemicals Co. (St. Louis, Mo.). Sodium dodecyl sulfate (Lot #11120KX) from Aldrich Chemical Co., Inc. Perchloric acid 70% (Lot #945567), chloroform HPLC grade (Lot #902521) and potassium phosphate monobasic (Lot #914723) (Fisher, Pittsburgh, Pa.).

The preparation of samples was carried out as follows: The SSM stock solution (1.59 mM) was prepared by weighing approximately 2.23 mg of DSPE-PEG₂₀₀₀ into a round bottom flask (RBF). Saline (˜0.5 ml) was added to achieve a concentration of 1.59 mM. The mixture was mixed with a vortex for 2 minutes at maximum speed. The solution was then flushed with argon and equilibrated in the dark at 25° C. for at least 1 h. The GLP-1 stock solution (176.67 μM≡582.56 μg/ml) was prepared by weighing approximately 12 μg of GLP-1 peptide. Saline (˜20 μl) was added to form the stock solution (176.67 μM). The VIP stock solution (176.67 μM≡587.57 μg/ml) was prepared by weighing approximately 12 μg of GLP-1 peptide. Saline (˜20 μl) was added to form the stock solution (176.67 μM). The exendin(9-39) stock solution (530 μM≡1786.0 μg/ml) was prepared by weighing approximately 108 μg of exendin(9-39) peptide. Saline (˜60 μl) was added to form the stock solution (530 μM). The VIP(6-28) stock solution (530 μM≡1492.6 μg/ml) was prepared by weighing approximately 90 μg of VIP(6-28) peptide. Saline (˜60 μl) was added to form the stock solution (530 μM). Lipid diluent (265 μM) was prepared and added to maintain critical micelle concentration (CMC) of DSPE-PEG₂₀₀₀ and prevent breaking of micelles. 0.1 ml of the SSM stock solution (1.59 mM) was diluted with 0.5 ml of saline to form the lipid diluent (265 μM). P. aeruginosa solution (strain PA103; 10⁵ cells/10 μl). Depending on the initial concentration of the P. aeruginosa suspension, cells were diluted to achieve a cell count of 10⁵ cells/10 μl.

Samples and controls were prepared as shown in Table 1 set out below; the samples and controls were incubated at 25° C. for 2 h in the dark prior to use in the experiments.

TABLE 1

| | | SSM | Final | | |
|---|---|---|---|---|---|
| | | stock | conc. of | GLP-1/VIP | Final conc. |
| | Saline | soln | SSM | stock soln | of GLP- |
| Ctrls/Samples | (μl) | (μl) | (mM) | (μl) | 1/VIP (μM) |
| Saline | 100 | — | — | — | — |
| SSM | 15 | 25 | 0.994 | — | — |
| GLP-SSM (GM) | — | 25 | 0.994 | 15 (GLP-1) | 66.25 |
| VIP-SSM (VM) | — | 25 | 0.994 | 15 (VIP) | 66.25 |

The cells were prepared as described below. Cell (10⁵) (˜1 ml) were plated into each well of a 12-well plate. This procedure was repeated until 22 wells were plated with cells. Cells were incubated for at least 6 h at 37° C., 5% CO₂ to allow cells to adhere to the culture plate. After 6 h, medium was removed and replaced with serum starved medium (with 2% FBS, and phenol red containing DMEM and P/S). Cells were incubated again for at least 6 h at 37° C., 5% CO₂.

TREM1 expression levels were determined as set out below. Before the addition of sample/control to the cells, the medium was removed from each well, cells were washed with PBS, and 0.5 ml of serum free medium (DMEM with no phenol red, FCS or antibiotics) was added into each well. The cells were treated according to Table 2 set out below. For cells in which the antagonist, Exendin(9-39) or VIP(6-28), was to be added, the respective receptor antagonist was added to the cells and left to incubate with the cells for 30 min at 37° C. before adding any other treatment. After the 30 min incubation, substances were added according to Table 2 set out below. For micelle-containing samples, lipid diluent was added before SSM/GM/VM. P. aeruginosa was added to the indicated cells 18 h after addition of peptides/SSM/saline.

TABLE 2

| | | | | | | | | | PA103 |
|---|---|---|---|---|---|---|---|---|---|
| | | | Lipid | | | | | VIP(6- | (μl)- |
| | | Saline | diluent* | SSM | GM | Exendin(9- | VM | 28) | add 18 h |
| Groups | Treatment | (μl) | (μl) | (μl) | (μl) | 39) (μl) | (μl) | (μl) | later |
| Controls | Saline | 30 | | | | | | | |
| | SSM | 20 | 2 | 8 | | | | | |
| | DSPE-PEG | 28 | 2 | | | | | | |
| | GM | 20 | 2 | | 8 | | | | |
| | VM | 20 | 2 | | | | 8 | | |
| | Ex(9-39) | 20 | | | | 10 | | | |
| | VIP(6-28) | 20 | | | | | | 10 | |
| | SSM + Ex(9-39) | 10 | 2 | 8 | | 10 | | | |
| | SSM + VIP(6-28) | 10 | 2 | 8 | | | | 10 | |
| | GM + Ex(9-39) | 10 | 2 | | 8 | 10 | | | |
| | VM + VIP(6-28) | 10 | 2 | | | | 8 | 10 | |
| PA103 | Saline | 20 | | | | | | | 10 |
| stimulated | SSM | 10 | 2 | 8 | | | | | 10 |
| | DSPE-PEG | 18 | 2 | | | | | | 10 |
| | GM | 10 | 2 | | 8 | | | | 10 |
| | VM | 10 | 2 | | | | 8 | | 10 |
| | Ex(9-39) | 10 | | | | 10 | | | 10 |
| | VIP(6-28) | 10 | | | | | | 10 | 10 |
| | SSM + Ex(9-39) | | 2 | 8 | | 10 | | | 10 |
| | SSM + VIP(6-28) | | 2 | 8 | | | | 10 | 10 |
| | GM + Ex(9-39) | | 2 | | 8 | 10 | | | 10 |
| | VM + VIP(6-28) | | 2 | | | | 8 | 10 | 10 |

*Lipid diluent (with a final concentration of 1 μM) was added before addition of SSM/GM/VM
Final conc. of GLP-1 = 1 μM
Final conc. of VIP = 1 μM
Final conc. of Exendin(9-39) = 10 μM
Final conc. of VIP(6-28) = 10 μM
Final conc. of SSM = 15 μM
PA103 = 10⁵ cells/10 μl (Multiplicity of infection = 1)

After substances were added, cells were incubated for another 24 h at 37° C. Culture medium was collected from each well. The remaining cells were washed twice with PBS, and cell lysis buffer (100 μl) (luciferase kit) was added. Cells were dislodged from the culture dish with a cell scraper and collected in centrifuge tubes. All samples were stored at −80° C. if not used immediately. The expression level of TREM 1 in cell lysate of each well was measured using a luciferase assay (Cat#1500, Promega). The protein content from each sample was measured using a Bradford protein assay to normalize results of the luciferase assay.

As observed in previous experiments, the saline-treated macrophages exhibited significantly higher luciferase activity when stimulated with P. aeruginosa compared to the saline control, indicating the induction of inflammation with higher expression levels of TREM 1. Similar luciferase activities were obtained with cells pre-treated with sub-micellar concentration of DSPE-PEG₂₀₀₀, the glucagon-like peptide-1 (GLP-1) receptor antagonist Exendin (9-39), and the vasoactive intestinal polypeptide (VIP) receptor antagonist VIP(6-28), demonstrating the absence of anti-inflammatory effect by these agents.

On the other hand, macrophages treated with SSM, GLP-SSM (GM) and VIP-SSM (VM) all displayed significantly lower luciferase activities (i.e., lesser inflammation) compared to the saline-treated P. aeruginosa-stimulated cells (p<0.05). This was indicative of lower expression levels of TREM 1 and hence potential anti-inflammatory effects of SSM, GM and VM. These observed anti-inflammatory responses were unaffected by the presence of Exendin (9-39) and VIP(6-28).

This experiment indicates that (1) SSM suppress the inflammatory response induced by P. aeruginosa on macrophages, (2) the anti-inflammatory effects of SSM are not mediated via the GLP-1 receptor or the VIP receptor, (3) a micellar concentration of DSPE-PEG₂₀₀₀ is required for the observed anti-inflammatory effects of SSM, and (4) that the anti-inflammatory effects of SSM could possibly be mediated via a direct interaction of SSM with P. aeruginosa bacteria.

It has been reported recently that phospholipid interacts with Gram-negative bacteria lipopolysaccharide (LPS) (Nomura et al., Biophys. J. 95: 1226-38, 2008). Such interaction could interfere with the binding of LPS with its accessory proteins (e.g. LPS-binding protein), resulting in the decreased interaction of LPS with its cell surface receptor, toll-like receptor 4 (TLR4), which is required for the induction of an inflammatory response (Bochkov et al., Nature 419: 77-81, 2002).

### Example 2

### Sterically Stabilized Micelles Reduce or Inhibit Endotoxin-Induced Activation of NF-κB in Macrophages

Host responses that occur during infection can be reproduced by administration of bacterial fragments, the most extensively studied of which is endotoxin (LPS) from Gram-negative bacteria. Lipopolysacharride (LPS), which is found in the circulation during sepsis, induces cytokine release, hypotension, and death. LPS also induces the metabolic responses seen during infection. To determine if sterically stabilized phospholipids micelles attenuate endotoxin-induced activation of pro-inflammatory mediators, the following experiment was carried out.

Bone marrow-derived macrophages (BMDM) from a primary macrophage cell line, extracted from mice transfected with a nuclear factor-kappa B (NF-κB)-driven luciferase reporter plasmid, were used in these experiments. In this cell line, the expression of NF-κB (a proinflammatory mediator) induces the expression of a luciferase gene in a concentration-dependent manner. Therefore, the expression level of NF-κB in BMDM can be quantified indirectly by the magnitude of luminescence produced [relative luminescence units (RLU)] via a luciferase assay. BMDM were subjected to treatment with either saline, saline+LPS, SSM, SSM+LPS, GLP, GLP+LPS, GLP-SSM (GM), GM+LPS, VIP, VIP+LPS, VIP-SSM (VM), and VM+LPS. The resulting inflammatory responses induced by the different agents was then quantified via a luciferase assay to determine the expression level of NF-κB.

The following reagents were used in the experiments: Murine BMDM with NF-κB-driven luciferase reporter construct [Source: Mice with NF-kappaB-driven luciferase reporter construct (HIV-LTR/luciferase; HLL)], Glucagon-Like Peptide I (7-36) (MW 3297.5, Cat#46-1-13B, American Peptide), VIP (Research Resources Center, the University of Illinois at Chicago), DSPE-PEG₂₀₀₀ (MW 2810, Cat#: PE 18:0/18:0-PEG 2000, Lot#899346-1/09, Lipoid), Saline, Cell Culture Medium: DMEM (Cellgro) containing 10% FCS (Hyclone), penicillin (100 U/ml)/streptomycin (100 μg/ml) (Invitrogen), DMEM with no phenol red (Cat#21063), PBS, Cellgro, E. coli LPS (Sigma-Aldrich), and Luciferase assay kits (Cat#1500, Promega).

Test reagents for use in the experiments were prepared using the following protocols as set out in detail below. SSM stock solution (1.56 mM): Weigh approximately 2.2 mg of DSPE-PEG₂₀₀₀ into a round bottom flask (RBF). Add the required volume of saline (˜0.5 ml) to achieve a concentration of 1.56 mM. Vortex the mixture for 2 minutes at maximum speed. Flush the solution with argon and equilibrate in the dark at 25° C. for at least 1 hour. GLP-1 stock solution (173.3 μM≡571.57 μg/ml): Weigh approximately 17.2 μg of GLP-1 peptide. Dissolve in the required volume of saline (˜30 μl) to form stock solution (173.3 μM). Test samples and controls were prepared as set out in Table 2, and samples and control were incubated at 25° C. for 2 h in the dark. VIP stock solution (176.67 μM≡587.57 μg/ml): Weigh approximately 12 μg of VIP peptide. Saline (˜20 μl) was added to form the stock solution (176.67 μM). LPS stock solution (LPS is weighed from lyophilized powder and dissolved in PBS; each well contains 1 μg/ml).

Preparation of cells for test: Extracted bone marrow cells were grown for 7 days in liquid culture medium (LCM) containing full medium. Old medium was removed and fresh medium (10-20 ml) was added. Cells were dislodged by scraping using a cell scraper, and the cells were counted. The concentration of cells was adjusted with medium to the test concentration of 10⁵ cells/ml. Cells (10⁵) (˜1 ml) were plated into each well of a 12-well plate. This procedure was repeated until 16 wells were plated with cells. Cells were incubated for 24 h at 37° C., 5% CO₂. This allowed cells to adhere to the culture plate. After 24 h, medium was removed and replaced with serum-starved medium (with 2% FBS, phenol red containing DMEM and Pen/Strep (P/S)). Cells were incubated at 37° C., 5% CO₂.

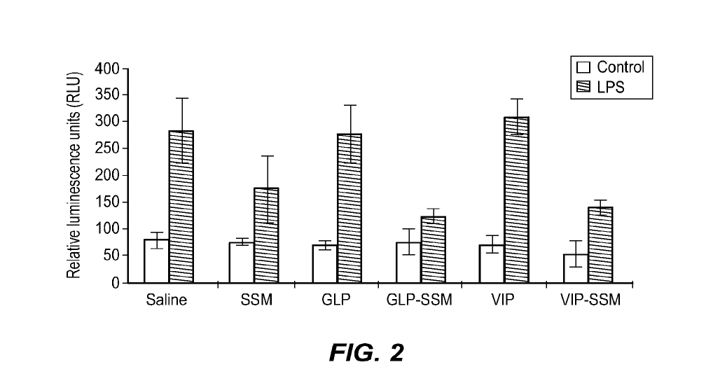

Determination of NF-κB expression level: Before addition of sample/control, the media was removed from each well; washed with PBS and 0.5 ml of serum free media (DMEM with no phenol red, FCS or antibiotics) was added into each well. Cells were treated as set out in Table 3 below. Cells were then incubated for 24 h at 37° C. The culture media was collected from each well. Remaining cells were washed twice with PBS; 100 μl of cell lysis buffer (luciferase kit) was added; cells were dislodged with a cell scraper and collected in centrifuge tubes. All samples were stored at −80° C. if not used immediately. The expression level of NF-kappa B (relative luminescence units (RLU)) in the cell lysate from each well was measured using a luciferase assay (Cat#1500, Promega). The protein content of each sample was measured using a Bradford protein assay to normalize results from the luciferase assay. The results are set out in Table 3 below and in FIG. 2.

TABLE 3

| | Relative Luminescence Units (RLU) Normalized to Protein Concentration | | | | | | | |
|---|---|---|---|---|---|---|---|---|
| | | | | | | | p | p |
| | | | | | | | value | value |
| | | | | | | | (vs | (vs |
| | Trial | Trial | Trial | Trial | | Std. | saline | SSM |
| Samples | 1 | 2 | 4 | 3 | Ave. | Dev. | LPS) | LPS) |
| Saline | 85.34 | 90.73 | 61.22 | 33.37 | 79.10 | 15.72 | | |
| (control) | | | | | | | | |
| Saline + LPS | 278.96 | 225.82 | 347.98 | | 284.25 | 61.25 | | |
| SSM | 71.05 | 84.53 | 76.50 | 71.05 | 75.78 | 6.38 | | |
| (control) | | | | | | | | |
| SSM + LPS | 113.96 | 219.26 | 129.16 | | 174.21 | 63.71 | 0.147 | |
| GLP | 64.92 | 85.24 | 65.18 | 64.92 | 70.06 | 10.12 | | |
| (control) | | | | | | | | |
| GLP + LPS | 336.41 | 225.11 | 272.28 | | 277.93 | 55.87 | 0.901 | |
| GLP-SSM | 109.79 | 79.34 | 55.28 | 65.25 | 77.41 | 23.73 | | |
| (control) | | | | | | | | |
| GM + LPS | | 136.50 | 115.36 | 144.54* | 125.93 | 14.95 | 0.042 | 0.56 |
| VIP | 57.78 | 90.09 | 86.45 | 60.50 | 73.70 | 16.92 | | |
| (control) | | | | | | | | |
| VIP + LPS | 329.26 | 342.42 | 267.10 | 306.81 | 311.39 | 32.99 | 0.478 | |
| VIP-SSM | 70.94 | 23.14 | 71.77 | 75.31 | 55.29 | 24.84 | | |
| (control) | | | | | | | | |
| VM + LPS | 158.11 | 149.33 | 126.49 | 139.91 | 143.46 | 13.53 | 0.006 | 0.72 |

*value extracted from HIV-LTR/luciferase; cells extracted from young mice

Results of the present experiments show that SSM alone can decrease or neutralize the effects of endotoxin as demonstrated by the effect that SSM had on the proinflammatory mediator NF-κB in the presence of endotoxin. Lipids such as SSM can apparently neutralize or inhibit the pro-inflammatory effect(s) of endotoxin. Such a use for SSM is especially valuable in the manufacture of and/or storage of recombinant proteins, wherein endotoxins from bacterial vectors can cause serious adverse events, or even death, in a mammal.

### Example 3

### Sterically Stabilized Micelles Attenuate Endotoxin-Induced Activation of NF-κB in Macrophages

Host responses that occur during infection can be reproduced by administration of bacterial fragments, the most extensively studied of which is endotoxin (LPS) from Gram-negative bacteria. LPS, which is found in the circulation during sepsis, induces cytokine release, hypotension, and death. LPS also induces the metabolic responses seen during infection. Lipoteichoic acid (LTA), a heat-stable component of the cell membrane and wall of most Gram-positive bacteria, has structural and functional similarities to LPS. Furthermore, LTA induces circulatory shock and treatment of macrophages or adherent mononuclear cells with LTA has been shown to induce cytokine mediators of septic shock (Bhakdi et al., Infect. Immun. 59:4614-4620, 1991). To further determine the effect that sterically stabilized micelles have on endotoxin, such as LPS and LTA, experiments are carried out with SSM, with and without GLP-1, in the presence of LPS, LTA, and Pseudomonas aeruginosa.

Bone marrow-derived macrophages (BMDM) from a primary macrophage cell line, extracted from mice transfected with a nuclear factor-kappa B (NF-κB)-driven luciferase reporter plasmid, are used in these experiments. In this cell line, expression of NF-κB (a proinflammatory mediator) induces the expression of a luciferase gene in a concentration-dependent manner. Therefore, the expression level of NF-κB in BMDM can be quantified indirectly by the magnitude of luminescence produced [relative luminescence units (RLU)] via a luciferase assay.

BMDM are subjected to 18 hr of treatment with either SSM, GLP-1, GLP-1-SSM, or saline control, followed by stimulation using lipopolysaccharide (LPS), lipoteichoic acid (LTA) and Pseudomonas aeruginosa. The resulting inflammatory response induced by the different agents is quantified using a luciferase assay on cell lysates to determine the expression level of NF-κB.

The following reagents are used in the experiments: Murine BMDM with NF-κB-driven luciferase reporter construct [Source: Mice with NF-kappaB-driven luciferase reporter construct (HIV-LTR/luciferase; HLL)], Glucagon-Like Peptide I (7-36) (MW 3297.5, Cat#46-1-13B, American Peptide), E. coli LPS (Sigma-Aldrich), LTA (Sigma Chemical Co., St. Louis, Mo.) DSPE-PEG₂₀₀₀ (MW 2810, Cat#: PE 18:0/18:0-PEG 2000, Lot#899346-1/09, Lipoid), Saline, Cell Culture Medium: DMEM (Cellgro) containing 10% FCS (Hyclone), penicillin (100 U/ml)/streptomycin (100 μg/ml) (Invitrogen), DMEM with no phenol red (Cat#21063), PBS, Cellgro, and Luciferase assay kits (Cat#1500, Promega).

Test reagents for use in these experiments are prepared using the following protocols as set out in detail below. SSM stock solution (1.56 mM): Weigh approximately 2.2 mg of DSPE-PEG₂₀₀₀ into a round bottom flask (RBF). Add the required volume of saline (˜0.5 ml) to achieve a concentration of 1.56 mM. Vortex the mixture for 2 minutes at maximum speed. Flush the solution with argon and equilibrate in the dark at 25° C. for at least 1 hour. GLP-1 stock solution (173.3 μM≡571.57 μg/ml): Weigh approximately 17.2 μg of GLP-1 peptide. Dissolve in the required volume of saline (˜30 μl) to form stock solution (173.3 μM). Test samples and controls were prepared as set out in Table 4 below, and samples and control were incubated at 25° C. for 2 h in the dark.

TABLE 4

| | | SSM | | | Final |
|---|---|---|---|---|---|
| | | stock | Final conc. | | conc. |
| | Saline | soln | of SSM | GLP-1 stock | of GLP-1 |
| Ctrls/Samples | (μl) | (μl) | (mM) | soln (μl) | (μM) |
| Saline | 100 | — | — | — | — |
| SSM | 15 | 25 | 0.975 | — | — |
| GLP-1 | 35 | — | — | 15 | 52 |
| GLP-1-SSM | — | 25 | 0.975 | 15 | 65 |

Lipid diluent (260 μM) is added to maintain critical micelle concentration (CMC) of DSPE-PEG₂₀₀₀ and prevent breaking of micelles: Dilute 0.1 ml of the SSM stock solution (1.56 mM) with 0.5 ml of saline to form the lipid diluent (260 μM). LPS solution (52 μg/ml): Depending on the initial concentration of LPS, dilute with saline to achieve 52 μg/ml. LTA solution (5.2 μg/ml): Depending on the initial concentration of LTA, dilute with saline to achieve 5.2 μg/ml. Pseudomonas aeruginosa (P.A.): Prepare a suspension of Pseudomonas diluted with saline to achieve 10⁵ cells/10 μl.

Preparation of cells for test: Grow extracted bone marrow cells for 7 days in LCM containing full medium. Remove old medium and add 10-20 ml of fresh medium, dislodge cells by scraping using a cell scraper and count the cells. The concentration of cells is adjusted with medium to the test concentration of 10⁵ cells/ml. Plate 10⁵ cells (˜1 ml) into each well of a 12-well plate. Repeat this procedure till 16 wells are plated with cells. Incubate cells for 24 h at 37° C., 5% CO₂. This allows cells to adhere to culture plate. After 24 h, remove media and replace with serum-starved media (with 2% FBS, phenol red containing DMEM and P/S). Incubate cells at 37° C., 5% CO₂.

Determination of NF-κB expression level: Before addition of sample/control, remove the media from each well; wash with PBS and add 0.5 ml of serum free media (DMEM with no phenol red, FCS or antibiotics) into each well. Treat the cells according to Table 3 below. For SSM-containing samples, add lipid diluent before SSM/GLP-1-SSM. LPS will only be added to the indicated cells 18 h after addition of peptides/SSM/saline.

TABLE 5

| | Treatment | | Lipid | | | | LPS/LTA/P.A. |
|---|---|---|---|---|---|---|---|
| | groups | Saline | diluent* | SSM | GLP-1 | GLP-1- | (μl) -add 18 h |
| | (triplicate) | (μl) | (μl) | (μl) | (μl) | SSM (μl) | later |
| CONTROL | Saline | 20 | — | — | — | — | — |
| | SSM | 10 | 2 | 8 | — | — | — |
| | Saline + LPS | 10 | — | — | — | — | LPS: 10 |
| | Saline + LTA | 10 | — | — | — | — | LTA: 10 |
| | Saline + P.A. | 10 | — | — | — | — | P.A.: 10 |
| | SSM + LPS | — | 2 | 8 | — | — | LPS: 10 |
| | SSM + LTA | — | 2 | 8 | — | — | LTA: 10 |
| | SSM + P.A. | — | 2 | 8 | — | — | P.A.: 10 |
| SAMPLES | GLP-1 | 10 | — | — | 10 | — | — |
| | GLP-1-SSM | 10 | 2 | — | — | 8 | — |
| | GLP-1 + LPS | — | — | — | 10 | — | LPS: 10 |
| | GLP-1 + LTA | — | — | — | 10 | — | LTA: 10 |
| | GLP-1 + P.A. | — | — | — | 10 | — | P.A: 10 |
| | GLP-1- | — | 2 | — | — | 8 | LPS: 10 |
| | SSM + LPS | | | | | | |
| | GLP-1- | — | 2 | — | — | 8 | LTA: 10 |
| | SSM + LTA | | | | | | |
| | GLP-1- | — | 2 | — | — | 8 | P.A.10 |
| | SSM + P.A. | | | | | | |

*Lipid diluent (with a final concentration of 1 μM) is added before addition of SSM/GTP-1-SSM.
Final GLP-1 conc. = 1 μM
Final SSM conc. = 15 μM
Final LPS conc. = 1000 ng/ml
Final LTA conc. = 100 ng/ml
Pseudomonas aeruginosa (P.A.): multiplicity of infection = 1

Cells are incubated for 24 h at 37° C., and the culture medium is collected from each well. The remaining cells are washed twice with PBS, 100 μl of cell lysis buffer (luciferase kit) is added. Cells are then dislodged with a cell scraper and collected in centrifuge tubes. All samples are stored at −80° C. if not used immediately. The expression level of NF-kappa B in the cell lysate from each well is measured using a luciferase assay (Cat#1500, Promega). The protein content of each sample is measured using a Bradford protein assay to normalize results of the luciferase assay.

Sterically stabilized micelles apparently interact with LPS to inhibit its pro-inflammatory effect, and thereby will reduce or inhibit endotoxin-induced activation of NF-κB in macrophages.

### Example 4

### The Development of a Sterically Stabilized Micellar Formulation of Polymyxin B

Polymyxin B (PxB) is a potent amphiphilic decapeptide antibiotic composed of a hydrophilic polar charged cyclic ring and a hydrophobic 8-carbon acyl chain. Unfortunately, PxB is not suitable for parenteral use in humans because it readily self-aggregates in aqueous solution, both in saline and HEPES Buffer (pH-7.4). To determine if PxB could be prepared in a sterically stabilized phospholipids micelle formulation for subsequent drug delivery in mammals, the following experiment was carried out.

PxB aggregates formed over the range of concentrations tested, from 10 μM to 23 mM. It was therefore speculated that when PxB is incubated with SSM, PxB forms aggregates, which might prevent PxB from interacting with or incorporating into the micelle. Therefore, in order to prevent PxB aggregates, DSPE-PEG₂₀₀₀ and PxB were co-precipitated together. This protocol is the same as the one described for incorporating hydrophobic drugs into SSM (see U.S. Pat. No. 6,217,886, incorporated herein by reference in its entirety). By creating a film of drug and lipid in a round bottom flask, upon re-hydration the drug molecules, i.e. PxB, interact directly with the DSPE-PEG₂₀₀₀, thereby either incorporating the drug into the micelle or associating the drug with the micelle.

In order to determine the optimum peptide to lipid ratio of PxB to SSM, fluorescence spectroscopy and NICOMP analysis were carried out on PxB at varying concentrations in sterile normal saline (SNS) and with DSPE-PEG₂₀₀₀ at a fixed concentration. In these experiments, PxB Sulphate (MW 1302 μg/mol, Research Products International Corp, Cat: P40160-1.0, Lot#003746), DSPE-PEG₂₀₀₀ (MW 2811 g/mol, Lipoid, Cat#PE18:0/18:0/PEG 2000, Lot#899346-1/10), Methanol, and HEPES Buffer (pH˜7.4) were used. Controls: SSM (1 mM) and Samples: PxB-SSM (1:1000) were prepared.

Additional solutions were prepared for the experiments as set out in detail below. Preparation of PxB Stock Solution (50 μM): Approximately 130.0 μg of PxB powder was weighed using the microbalance and then transferred into a 2 ml vial. PxB was then dissolved in 2 ml of methanol to form a 50 μM PxB solution and vortexed for 2 minutes. Preparation of the SSM Stock Solution (2 mM): 2.81 mg of DSPE-PEG₂₀₀₀ was weighed and dissolved in 1 ml of methanol; this solution was then vortexed for 2 minutes. Preparation of PxB-SSM solution for co-precipitation. The corresponding volumes of PxB, DSPE-PEG₂₀₀₀, and methanol were added to 100 ml round bottom flasks as indicated in Table 6 below.

TABLE 6
PxB-SSM Co-Precipitation Solutions

| | | Volume of | Volume of | Total |
|---|---|---|---|---|
| | Volume of PxB | SSM solution | methanol | Volume |
| Sample | solution (μl) | (μl) | (μl) | (μl) |
| SSM_{control} | 0 | 250 | 250 | 500 |
| PxB_{1 μM}-SSM | 10 | 250 | 240 | 500 |
| PxB_{5 μM}-SSM | 50 | 250 | 200 | 500 |
| PxB_{10 μM}-SSM | 100 | 250 | 150 | 500 |
| PxB_{15 μM}-SSM | 150 | 250 | 100 | 500 |
| PxB_{1 μM} | 10 | 0 | 490 | 500 |
| PxB_{5 μM} | 50 | 0 | 450 | 500 |
| PxB_{10 μM} | 100 | 0 | 400 | 500 |
| PxB_{15 μM} | 150 | 0 | 350 | 500 |

Fluorescence Spectroscopy (SLM Aminco 8000): Configurations were set for sterile water detection in the Fluorometer. Channel A: Gain=100; HV=1200; type=slow; Integration=1; Excitation=350 nm, step=1 nm; Emission=397 nm, step=0.2 nm; Band Pass=8 for emission and excitation. The peak intensity should be at 397±0.5 nm. Each sample was placed into the quartz cell. Channel A: Gain=100; HV=1200; type=slow; Integration=1; Excitation=256 nm, step=1 nm; Emission=282 nm, step=1 nm; Band Pass=4 for emission and excitation; Ex Res=4 nm, Em Res=4 nm, X-axis=270 to 400 nm. NICOMP Analysis was carried out for each of the samples.

Specifically, PxB (at various concentrations ranging from 0.5 mM to 6.9 mM) in sterile normal saline (SNS) was incubated with DSPE-PEG₂₀₀₀ (1 mM). Stock solutions of PxB and DSPE-PEG₂₀₀₀ were prepared and equilibrated for 2 hours. After the 2 hour equilibration, stock solutions were characterized by dynamic light scattering (DLS) using the NICOMP 380 Submicron Particle Sizer (Particle Sizing Systems, Inc. Santa Barbara, Calif.). After the characteristic peaks were observed in the stock solutions, the sample solutions were prepared (time=0). A SSM sample (1 mM) (n=1) was prepared in saline alone for comparison with the PxB-SSM samples. Concurrent samples of PxB (0.5, 1.0, 2.3 and 6.9 mM) were prepared in saline alone or with SSM (DSPE-PEG₂₀₀₀, 1 mM) (n=8). The samples were measured for 30 minutes using DLS at approximately 2, 24, and 48 hours after time 0.

SSM (n=1):

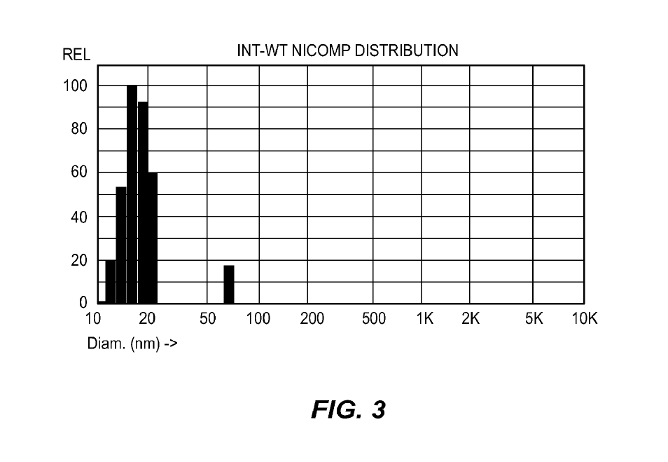

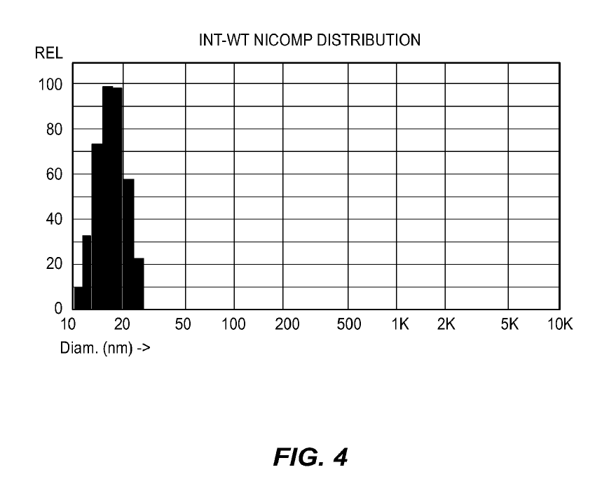

The 1 mM DSPE-PEG₂₀₀₀ solution remained stable for at least 48 hours after preparation and produced a singular peak at approximately 15 to 17 nm in diameter (FIGS. 3 and 4).

PxB (6.9 mM) (n=2 and 3):

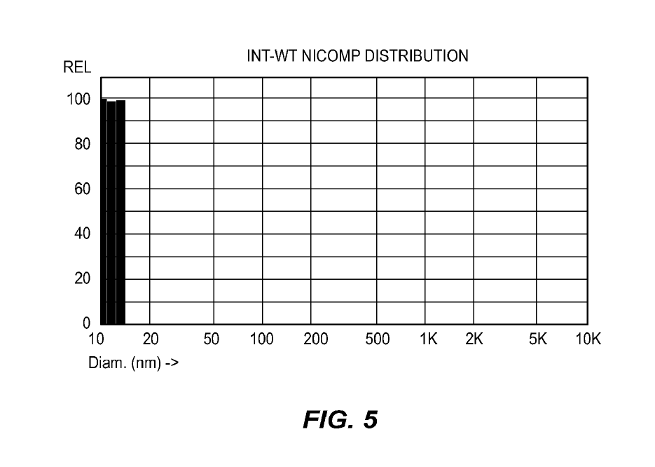

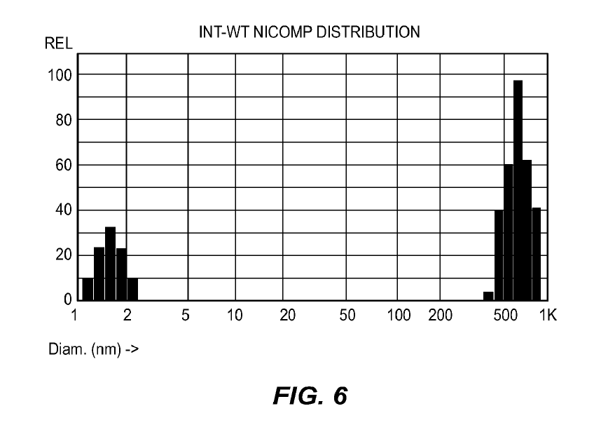

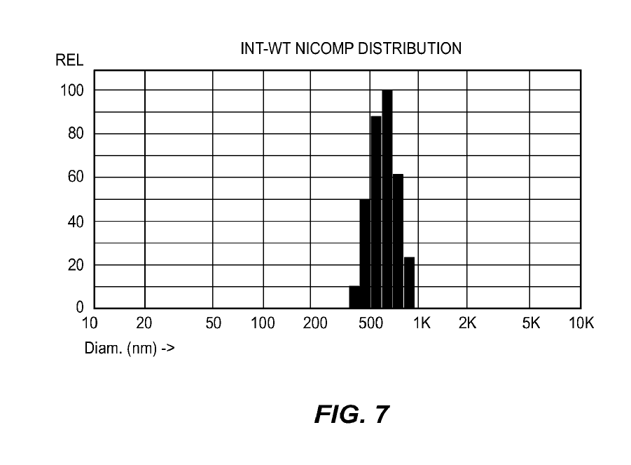

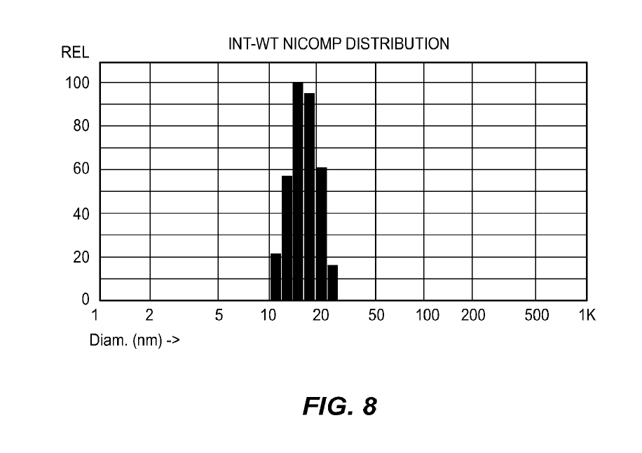

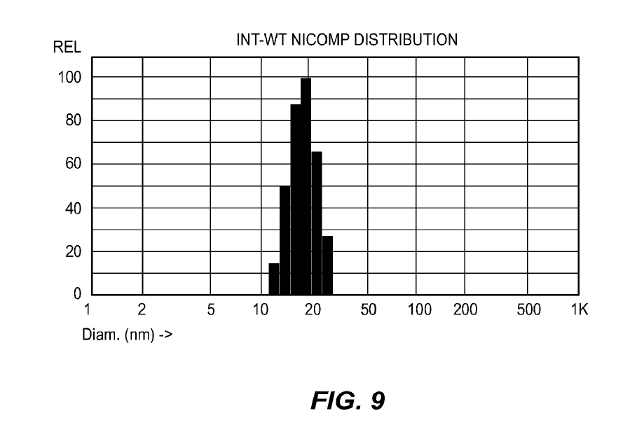

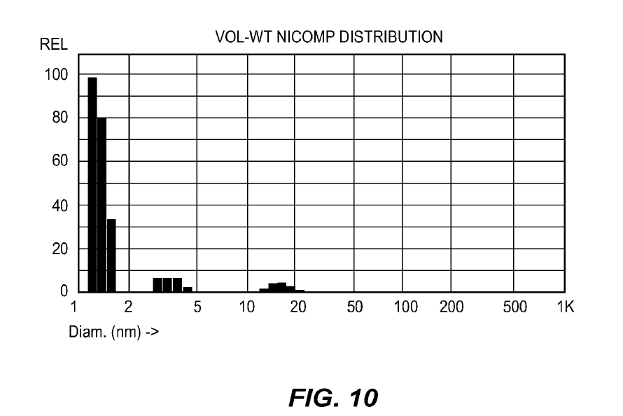

PxB (6.9 mM) in saline alone (n=2) did not show peaks in the graph from 10 to 10,000 nm (FIG. 5), however when the range was set between 1 and 1,000 nm (FIG. 6), large particles were detected at approximately 640 nm with smaller particles at 1 nm in diameter as well. The presence of these particles was further confirmed at the 24 and 48 hour time points (FIG. 7) with an average particle size of 517 nm in diameter. In the solution containing both PxB (6.9 mM) and DSPE-PEG₂₀₀₀ (n=3), no large aggregates were observed. The only detectible peaks were between 12 and 18 nm in diameter, corresponding to the micellar peak, along with smaller particles below 5 nm (FIG. 8). This phenomenon was seen before in a previous experiment and recorded in the previous report. At the 48 hour time point (FIGS. 9 and 10), a third particle size was detected between 3 and 4 nm in diameter. At the final time point, the 1 nm particles comprised 90% of the sample, 3-4 nm particles comprised 6% of the particles detected, and the SSM comprised 4% of detected particles.

PxB (2.3 mM) (n=4 and 5):

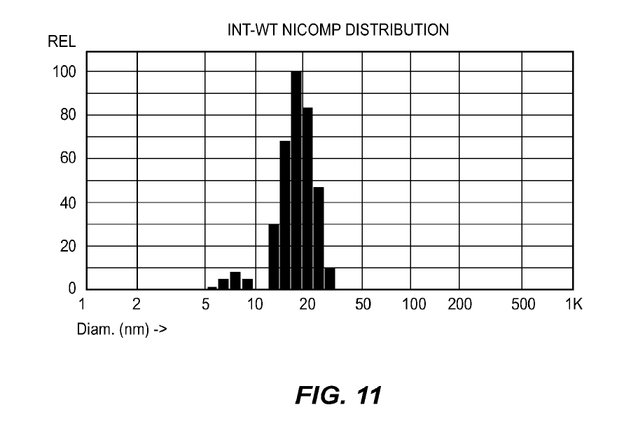

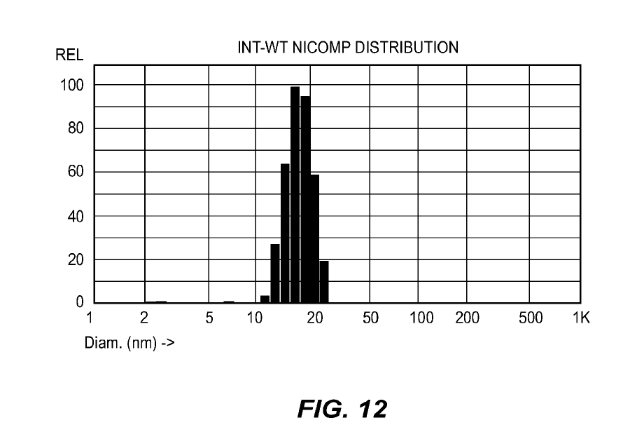

The same effect in the initial measurement of PxB in saline alone that occurred in n=2 happened in n=4 (FIGS. 5 and 6) with the difference being the size of the aggregate formed, approximately 850 nm. The large aggregates were observed at the 24 and 48 hour time points as well. In the PxB-SSM sample (n=5), two particle sizes were observed after 2 hours of equilibration at 7 and 20 nm (FIG. 11), but at the 24 and 48 hour time points, the 7 nm particle decreased to 2.4 nm (FIG. 12). No large aggregates were observed at any of the time points when 2.3 mM PxB was incubated with 1 mM DSPE-PEG₂₀₀₀. At the final time point, the 2.4 nm particles comprised 74% of the sample and the SSM comprised 26% of detected particles.

PxB (1.0 mM) (n=6 and 7):

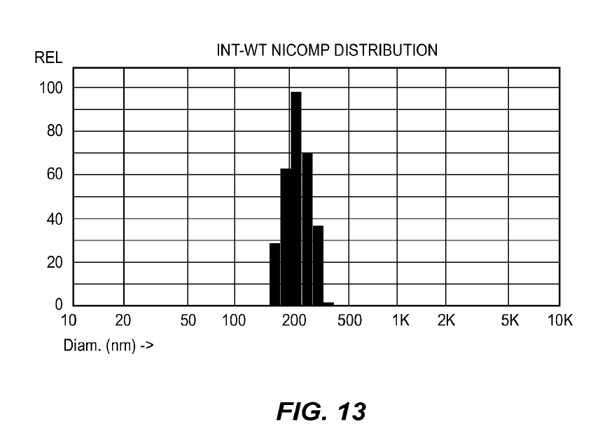

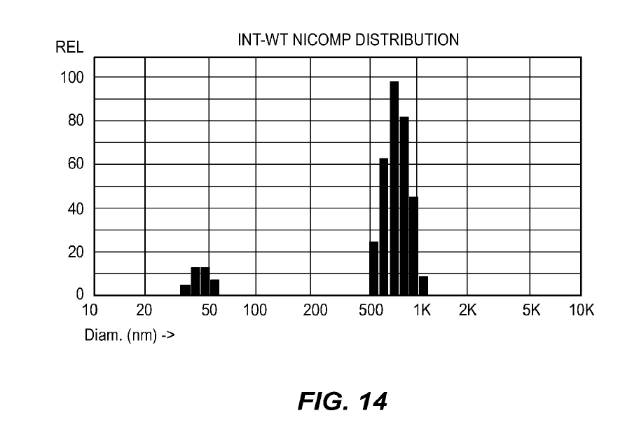

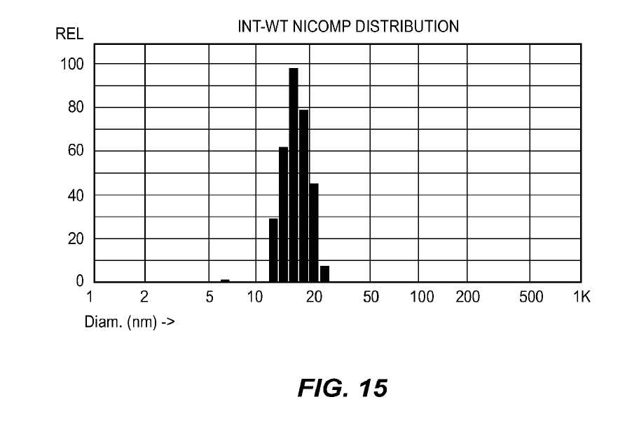

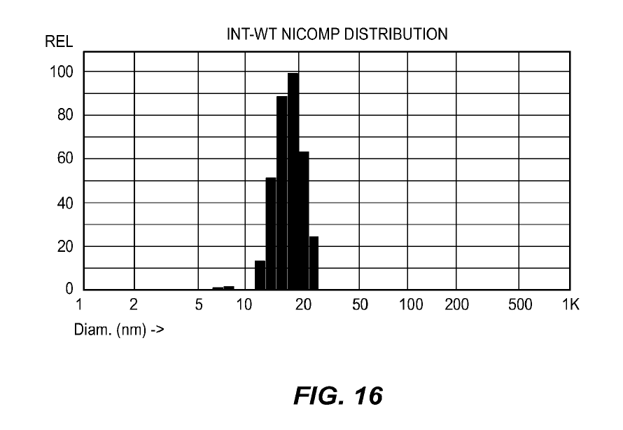

The 1 mM PxB in saline alone (n=6) provided the most consistent data at each time point. After two hours of equilibration, aggregates of 240 nm were observed (FIG. 13). By the 24 hour time point the aggregate reached a size of 520 nm, which further increased to 726 nm and the appearance of smaller particles of approximately 45 nm (FIG. 14). For the each time point, the detectible particles in the PxB-SSM sample (n=7) corresponded to the DSPE-PEG₂₀₀₀ micellar size (FIG. 15, time=2 hours) and a second peak at 7 nm in diameter (FIG. 16, time=48 hours). There were no large aggregates in the PxB (1 mM) in SSM sample. At the final time point the 7 nm particles comprised 29% of the sample and the SSM comprised 71% of detected particles.

PxB (0.5 mM) (n=8 and 9):

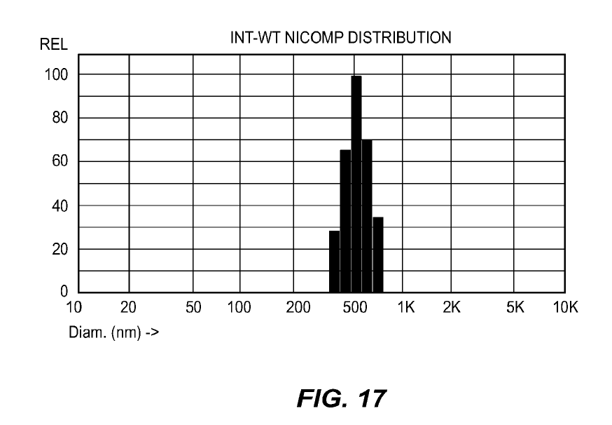

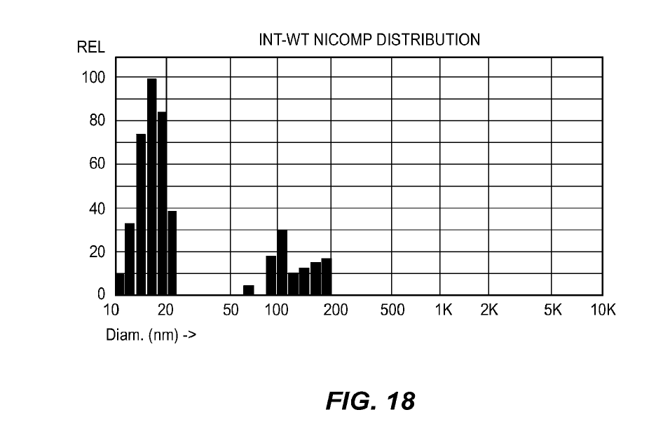

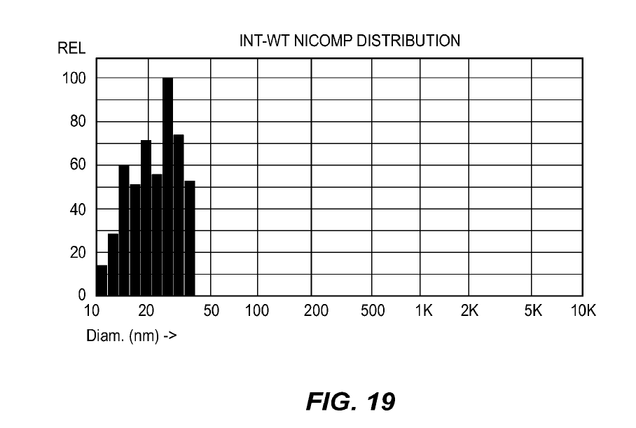

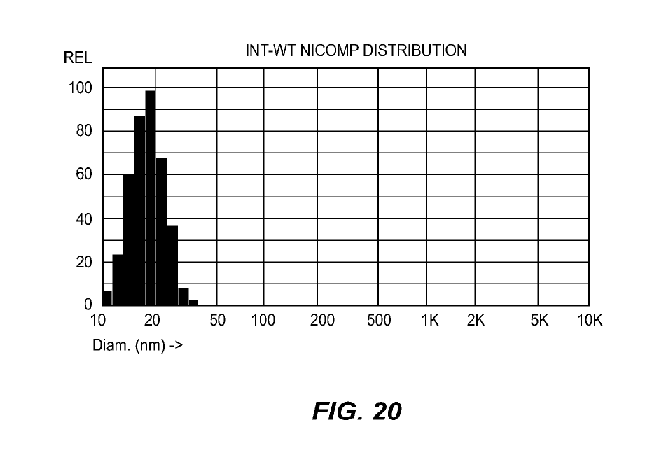

The 0.5 mM PxB in saline alone (n=8) had the same phenomenon occur as the first two samples (n=2 and 4) produced not only the large aggregates and small 1 nm particles, but also particles at approximately 7 nm in diameter at the two hour time point. By the 24 hour time point, aggregates of approximately 520 nm had formed and remained the same in the 48 hour time point (FIG. 17). Two hours after the PxB (0.5 mM) was incubated with the 1 mM DSPE-PEG₂₀₀₀ the characteristic micelle peaks were observed with a larger particle above 100 nm (FIG. 18). After 24 hours the large 100+ nm particle had disappeared, but smaller particles appeared between 20 and 50 nm along with the peak of the SSM (FIG. 17). And at the final 48 hour time point, the only detectable peak was that of the SSM (FIG. 18). At the final time point, the entire solution showed only the SSM and neither the large 100+ nm aggregate or the 20 to 50 nm sized particles as seen in FIGS. 19 and 20.

The concentrations of PxB tested in SNS alone varied from 0.5 to 6.9 mM. This range included concentrations below (0.5 mM), at (1.0-2.3 mM), and above (6.9 mM) the critical micelle concentration (CMC) of PxB. Concurrent samples of PxB in saline alone as well as with a standard 1.0 mM DSPE-PEG₂₀₀₀ solution were prepared and were allowed to equilibrate for 2, 24, and 48 hours, at which times they were characterized by dynamic light scattering. All of the PxB in saline alone samples (n=2, 4, 6, and 8) showed aggregation of the peptide, even below the CMC. Conversely, in all of the samples in which PxB was incubated with DSPE-PEG₂₀₀₀ (n=3, 5, 7, and 9), no large aggregates were observed. These results confirm what was observed in the previous experiment by the free peptide aggregating as well as by destabilizing the aggregates and by preventing further peptide aggregation.

The volume weighting distribution of the PxB-SSM samples showed the relative number of particles of each size in the sample. In each successive sample, the number of PxB particles compared to the number of SSM decreased, as the concentration of PxB decreased. At 0.5 mM PxB with 1.0 mM DSPE-PEG₂₀₀₀, no small particle or PxB residual was observed with only the SSM peak being detectible.

PxB (0.5 mM) with 1.0 mM DSPE-PEG₂₀₀₀ (n=9) provided the best visualization of what occurs when PxB interacted with the lipid micelles. The PxB in saline alone sample showed that the peptide normally aggregates to a size of approximately 520 nm after 24 hours. At this concentration, the largest aggregate that formed was 520 nm. Larger particles or aggregates were not seen, and must not be able to form at this concentration or at a lower concentration due to a limit in the number of peptides that are available. Two hours after PxB was equilibrated with SSM, the large 520 nm particle disappeared but was replaced by one at 100 nm. This result indicates that the PxB aggregates are destabilized by SSM. The result of this destabilization is seen at the 24 hour time point in which PxB remains at a size between 20 and 50 nm in diameter. These particles disappeared by the 48 hour time point leaving only the clean SSM peak in the NICOMP graph, which shows that PxB must not only be interacting with the micelle but also must be associating with the SSM in some manner.

In summary, results from the experiments showed that PxB (0.5-6.9 mM) formed aggregates when dissolved in saline at all concentrations tested. The PxB aggregates ranged from 200 to 1500 nm in diameter and remained stable for at least 48 h at 25° C. By contrast, PxB-SSM exhibited reproducible size in saline (14-17 nm) and prevented the formation of PxB aggregates at all concentrations tested (0.5-6.9 mM). Importantly, the PxB-SSM suspension remained stable for at least 48 h at 25° C. These data indicate that sterically stabilized phospholipid nanomicelles constitute a novel, long-acting, biocompatible and biodegradable nanocarrier for PxB. Accordingly, the invention provides PxB-SSM. This compound should be further developed as an anti-infective drug in the treatment of infection resulting from resistant bacteria.

### Example 5

### Determining the DSPE-PEG₂₀₀₀:Polymyxin B Saturation Ratio via Fluorescence Spectroscopy

To determine the saturation point for DSPE-PEG₂₀₀₀ with PxB, fluorescence spectroscopy was carried out. PxB concentration was fixed at 50 μM and the concentration of DSPE-PEG₂₀₀₀ was varied to allow DSPE-PEG₂₀₀₀:PxB ratios of 1:1, 3:1, 6:1, 10:1, 20:1, 60:1, and 100:1. A stock solution of DSPE-PEG₂₀₀₀ was prepared and allowed to equilibrate at 25° C. in the dark under argon gas for two hours. A stock solution of PxB (100 μM) was prepared in HEPES Buffer. Samples were prepared by pipetting the appropriate amount of buffer and stock solutions into each vial. Fluorescence measurements were made after allowing each sample to equilibrate for 1 hour in the dark at 25° C. 400 μl of each sample was measured three times by the spectrofluorometer. The maximum intensity of each sample was recorded from the spectrofluorometer and analyzed using Microsoft Excel and Sigma Plot.

To fit a curve to the graph of the data the equation

$$y = {y}_{0} + \frac{ax}{b + x}$$

was used because its shape best fit the data, where

y₀=0.9876, a=1.3979, b=5.1608

When the lipid to peptide ratio approaches infinity, a plateau is reached in terms of the fluorescence intensity and the equation becomes

$${y}_{∞} = {y}_{0} + \frac{ax}{x} = {y}_{0} + a = {y}_{p}$$

where yₚ is the height of the plateau. Because the lipid to peptide ratio will never reach infinity, it can be assumed that the plateau can be reached at approximately 90% of a,

which corresponds to,

yₚ=y₀+0.9a=2.24571

The lower limit of this value will be reached at a point which corresponds to the average standard deviation of all the samples,

y=yₚ−0.10165=2.14406

Using this value as y, the lipid to peptide saturation point can be calculated by substituting the values into the equation and solving for x,

$$x = \frac{( y - {y}_{0} ) b}{a - y + {y}_{0}} = 24.7$$

Therefore, the lipid to peptide ratio at the saturation point is 24.7:1, which corresponds to 3.6 molecules of PxB molecules per micelle.

### Example 6

### Optimizing Formulation of GLP-1 in SSM

The objective of these experiments was to determine the optimal formulation of GLP-1(7-36) in SSM (with the maximum peptide loading) by characterizing the interaction of GLP-1(7-36) with SSM in aqueous medium for delivering enzyme labile GLP-(7-36) in SSM to increase its in vitro and in vivo stability.

SSM composed of poly(ethylene glycol-2000)-grafted distearoylphosphatidylethanolamine (size, 15 nm), DSPE-PEG₂₀₀₀ phospholipid, were prepared as previously described (Ashok et al., J. Pharm. Sci. 93: 2476-87, 2004). Weighted amount of DSPE-PEG₂₀₀₀ was dissolved in saline, vortexed until complete dissolution and equilibrated for 1 hr at 25° C. in the dark. A measured volume of human GLP-1(7-36) peptide stock solution (in saline) was added to SSM or saline and incubated for 2 hr at 25° C. to achieve the desired peptide and/or lipid concentrations. The interaction of peptide with SSM was analyzed by circular dichroism, fluorescence spectroscopy, and fluorescence anisotropy.

Fluorescence spectroscopy. The samples prepared contained 5 μM of GLP-1(7-36) in saline or varying concentrations of SSM (0.0075 mM to 0.4 mM) to achieve lipid:peptide molar ratios varying from 0 to 80. The fluorescence emission spectra of samples were measured using SLM Aminco 8000 Spectrofluorimeter [Exλ (nm)/Emλ (nm): 275/340]. Self-association of GLP-1(7-36) with SSM was confirmed by a significant increase in the peptide fluorescence emission with a concomitant blue shift in peak wavelength (350 nm to 335 nm) compared to that of GLP-1(7-36) in saline, which is indicative of the peptide residing in a relatively more hydrophobic environment (SSM) than saline. Significant increase (p<0.05) in the emitted fluorescence intensity was observed for GLP-1(7-36) in SSM compared to the peptide in saline (n=3). Correspondingly, the peak wavelength of the fluorescence spectra showed a blue shift (350 nm to 335 nm) for GLP-1(7-36) in SSM relative to saline, indicating a change in environment from hydrophilic (saline) to relatively more hydrophobic (SSM). Based on the lipid:GLP-1 (7-36) saturation curve that was generated, a saturation molar ratio of 15:1 was determined. Given that approximately 90 lipid monomers form one micelle (Arleth et al., Langmuir 21:3279-90, 2005), it was thereby calculated that a maximum of six GLP-1(7-36) molecules could associate with one SSM.

Fluorescence anisotropy. The fluorescence anisotropy values of samples [GLP-1(7-36) (125 μM) in SSM (4.5 mM) or saline] were measured using Perkin Elmer Luminescence Spectrometer LS50B [Exλ (nm)/Emλ (nm): 275/340]. A significantly higher fluorescence anisotropy (p<0.05) was recorded for GLP-1(7-36) in SSM (0.108±0.009%; n=3) relative to saline (0.045±0.012 n=3). The data indicated an increased viscosity of the peptide surrounding in the presence of SSM in contrast to saline.

Circular dichroism. Spectra of samples [GLP-1(7-36) (20 uM) in SSM (5 mM) or saline] were scanned at room temperature in a 0.1 cm path length fused quartz under the following conditions: 190 to 260 nm at 1 nm bandwidth and 2 s response time averaged over 3 runs. Deconvolution of Spectra was performed using SELCON® to calculate the percentage of alpha-helical structures. The alpha-helicity of GLP-1(7-36) increased significantly (p<0.05) in the presence of SSM (33±7%; n=3) compared to that in saline (11±1%; n=3). The observed enhancement in the alpha-helicity of GLP-1(7-36), when associated with SSM, is highly desirable as it is the optimal peptide secondary conformation reported for ligand-receptor interaction (Runge et al., Biochemistry 46: 5830, 2007).

Human GLP-1(7-36) self-associates with SSM in aqueous media, as shown by the enhanced fluorescence emission and anisotropy of the peptide in the presence of SSM in comparison to saline. It was determined that a maximum of six GLP-1(7-36) molecules could self-associate with one micelle in the optimal formulation. The associated GLP-1(7-36) peptide also exhibited increased alpha-helicity, which is the optimal conformation reported for ligand-receptor interaction. SSM may be acting as a steric barrier to protect the associated peptide from enzymatic inactivation in vivo. These experiments indicate that human GLP-1(7-36) in SSM can be used as a novel nanomedicine in the treatment of diabetes and other inflammatory diseases.

### Example 7

### Anti-Inflammatory Activity of GLP-1 in SSM in a Model of Acute Lung Injury

To determine the effect of SSM comprising glucagons-like peptide-1 (GLP-1) in treating acute lung injury (ALI), SSM with and without GLP-1 was used to treat a murine model with ALI (ALI mice). Mice are induced with acute lung injury (ALI) by treatment with lipopolysaccharide (LPS) nebulization (as described by Sadikot et al. Am. J. Respir. Cell Mol. Biol. 164: 873-8, 2001; Koay et al., Am. J. Respir. Cell Mol. Biol. 26: 572-8, 2002). [PLEASE CONFIRM THIS IS CORRECT.]

ALI mice were divided into four groups (n=5) as follows: (1) saline+LPS (S+LPS); (2) GLP-1 (7-36)+LPS (G+LPS); (3) GLP-1-SSM+LPS (GM+LPS); and (4) an additional SSM control group, SSM+LPS (M+LPS). A dose of drug was administered subcutaneously (s.c.) 30 min after initialization of aerosolized LPS nebulization. When given, the dose of GLP-1(7-36) used was 15 nmol/mouse and SSM was 0.45 μmol/mouse (at a lipid concentration of 4.5 mM). All mice were sacrificed 4 hr after completion of nebulization. For each animal, bronchoalveolar lavage was carried out and the lungs were removed for analysis. Data from untreated (control) mice (no LPS nebulization or drug treatment) was also included in the data analysis for comparison.

Neutrophil cells were counted in bronchoalveolar lavage (BAL) fluid. Mice subjected to aerosolized LPS exhibited higher neutrophil cell count relative to the LPS-untreated controls. Among the LPS-exposed mice, neutrophil cell count was significantly lower (p<0.05) in GM+LPS mice compared to S+LPS, G+LPS, and M+LPS.

Neutrophilic enzyme activity in tissue was measured by a myeloperoxidase (MPO) assay of lung tissue. The MPO assay measures the magnitude of neutrophilic enzyme activity in tissue. As seen with neutrophil cell count in BAL fluid, significantly lower (p<0.05) MPO activity was measured in the lung tissue of GM+LPS-treated ALI mice compared to all other treatment groups, except for controls (untreated mice not exposed to LPS nebulization or drug treatment).

GM+LPS treatment decreased lung inflammation in ALI mice, as demonstrated by a significantly lower neutrophil cell count in BAL fluid and MPO activity of lung tissue compared to S+LPS, G+LPS, and M+LPS.

Quantification of cAMP by ELISA showed a significantly greater (p<0.05) level of cAMP in BAL fluid of GM+LPS mice compared to S+LPS, G+LPS, and M+LPS. These results suggest a possible role for cAMP-dependent cellular pathway in the observed anti-inflammatory effect of GM+LPS. Because GLP-1(7-36) receptor (GLP-1R) is a G protein-coupled receptor known to stimulate downstream adenylate cyclase and increase cAMP production when activated, the result also reaffirmed that the SSM delivered GLP-1(7-36) peptide retained its biological activity/ability to interact with its own specific receptor.

NF-KB, a pro-inflammatory transcription factor, was measured in the mouse lung tissue using a luciferase assay. Luciferase activity of the lung tissue was proportional to NF-KB activity. Results of this assay showed that there was no difference in NF-KB expression among the studied groups. Likewise, no significant differences were found in expression of downstream proinflammatory cytokines and chemokines (tumor necrosis factor-alpha (TNF-α) and leukotriene B4 (LTB4). It is possible that longer or repeated treatment with GLP-1-SSM may be needed for inhibition of the NF-KB-related inflammatory cascade. Likewise, it is possible the GLP-1-SSM inhibition of neutrophilic infiltration in the lung may occur via an NF-KB-independent mechanism.

Blood glucose levels were measured in mice from untreated controls (UC), S+LPS, G+LPS, and GM+LPS groups. Blood was taken via cardiac puncture at the conclusion of the experimental treatment. UC were not exposed to LPS nebulization or drug treatment. No significant increase in blood glucose levels was observed with LPS nebulization or drug treatment. Median blood glucose concentrations for S+LPS, G+LPS, GM+LPS, and UC groups were 183, 157, 125, and 109 mg/dL, respectively. To determine if GLP-1 SSM has a glucose regulating effect, a different animal model with hyperglycemia (db/db diabetic mice) is needed.

### Example 8

### Optimizing GLP-1(7-36) in SSM and SSMM

To optimize nanomicellar formulation of the peptide to be used in all subsequent experiments, interactions between GLP-1(7-36) and simple and mixed nanomicelles (SSM and SSMM, respectively), were elucidated. The GLP-1(7-36) peptide molecule has a hydrophilicity/hydrophobicity balance of ˜0.987. Based on experience with other amphipathic peptides, it was postulated that GLP-1(7-36) would associate spontaneously with SSM and SSMM in aqueous media. To test this hypothesis, fluorescence spectroscopy was used to measure peak fluorescence intensity of intrinsic fluorophores within the GLP-1(7-36) primary sequence in the presence and absence of nanomicelles. In addition, another goal of this experiment was to determine and compare the respective loading capacity of SSM and SSMM for GLP-1(7-36), which would indicate a maximum quantity of peptide that could be prepared per unit volume of SSM and SSMM dispersion.

With a fixed peptide concentration, increasing amount of lipid (prepared as SSM or SSMM dispersion) was added to achieve a range of varying lipid: peptide molar ratios. After 2 hr incubation at 25° C., peptide intrinsic fluorescence was measured using a spectrofluorometer (SLM-AMINCO Instruments, Inc., Rochester, N.Y.). In the presence of SSM and SSMM, GLP-1(7-36) exhibited increased fluorescence compared to peptide in saline. This phenomenon was possibly accountable by reduced quenching of peptide fluorophores when GLP-1(7-36) associated with nanomicelles. However, free peptide molecules are susceptible to collisional fluorescence quenching by surrounding water molecules and aggregated peptide fluorophores. A blue shift in peak wavelength of GLP-1(7-36) was observed in the presence of SSM and SSMM, signifying increased hydrophobicity of peptide local environment and hence providing further evidence of interaction between GLP-1(7-36) and nanomicelles. With a constant peptide concentration, increasing lipid amount initially increased and then eventually resulted in leveling off of peptide emission fluorescence when lipid content became excessive. The lowest molar ratio at which emitted fluorescence remained insignificantly different from fluorescence intensity at plateau was considered to be the lipid:peptide saturation ratio. Based on their respective lipid:peptide saturation curves, saturation ratios were 13:1 in SSM and 17:1 in SSMM for GLP-1(7-36). Given that approximately 90 lipid monomers form one nanomicelle (Ashok et al., 2004, supra), it was calculated that approximately 5 to 6 GLP-1(7-36) peptide molecules associated with each SSM or SSMM.

GLP-1(7-36) exists predominantly as a random coil in aqueous media and shows increased α-helicity in a hydrophobic environment with algorithm prediction. Therefore, to determine whether the peptide exhibits similar structural changes when incubated with phospholipid nanomicelles (SSM and SSMM), circular dichroism (CD) spectroscopy was carried out to measure peptide conformation in saline, SSM, and SSMM, respectively. Twenty μM of GLP-1(7-36) was added to saline, SSM or SSMM (5 mM), incubated as set out above and analyzed using a spectropolarimeter (J-710, Jasco Inc., Easton, Md.). Deconvolution of spectra was done by fitting data into simulations using the SELCON® program to calculate percentage of α-helical structures. There was a significant increase in α-helicity of GLP-1(7-36) in both SSM and SSMM, compared to peptide in saline. No significant difference was found between SSM and SSMM, corroborating fluorescence data that shows that the peptide interacts spontaneously with nanomicelles. Moreover, comparable structural changes of the associated peptides in SSM and SSMM also indicate that there is a similar interaction with the peptide molecules and each of the two nanomicellar systems, SSM or SSMM.

On the basis of the fluorescence and CD spectroscopy data, it was postulated that GLP-1(7-36) peptide molecules associate with SSM and SSMM at similar sites. This hypothesis was studied via determination of peptide fluorimetric anisotropy in the presence and absence of nanomicelles. This technique measures rigidity (or rotational freedom) of fluorophores by intensity of emitted fluorescence polarized parallel and perpendicular to that of excitation beam. A molecule that is restricted in its rotational motion due to viscous environment will emit fluorescence that is predominantly parallel to its excitation light source, giving a high anisotropy value. Experiments showed significantly higher anisotropy values in SSM compared to peptide in saline. Likewise, anisotropy of GLP-1(7-36) was not significantly different in the presence of SSM or SSMM. Therefore, these data show that the local environment surrounding the peptide fluorophores was of similar viscosity in SSM and SSMM, suggesting similar sites of interaction.

Aqueous formulations of self-associated peptide drugs with SSM are stable for only seven days at 25° C. which precludes prolonged storage before clinical use. To address this need for longer storage, SSM have been successfully lyophilized in the absence of additional cryo- and lyo-protectants. Moreover, physico-chemical properties of these nanomicelles are preserved upon reconstitution in aqueous media.

### Example 9

### Freeze Drying GLP-1(7-36) in SSM and SSMM

To determine if GLP-1-SSM formulation is sufficiently robust to withstand temperature and pressure changes that occurred during freeze drying, the following experiments were carried out. GLP-1-SSM was frozen overnight at −20° C., then incubated for 3 min in liquid nitrogen followed by lyophilization in Labconco® FreeZone Freeze Dryer (Labconco Corp., Kansas City, Mo.). The freeze dried samples were removed 24 hr later, examined visually, and reconstituted by addition of sterile water with gentle swirling. Visually, lyophilized cakes of GLP-1-SSM looked similar to blank SSM with similar time required for complete dissolution upon reconstitution (˜2 min). Moreover, particle size of peptide-associated SSM was comparable to that of empty SSM and did not change significantly pre- and post-lyophilization. Therefore, addition of peptide to SSM did not affect the freeze drying ability of the nanomicellar formulation.

For self-associated GLP-1(7-36) (67 uM) in SSM (10 mM), its fluorescence spectra showed similar magnitude of Emmax and corresponding peak wavelengths before and after freeze drying. Because there were no significant changes in fluorescence spectra after lyophilization, the experiments indicate that peptide interaction with SSM is not disturbed even after relatively harsh treatment of freeze drying. Consequently, the extent of peptide-nanomicelle interaction was not significantly affected by lyophilization. Likewise, percent α-helicity of self-associated GLP-1(7-36) (67 uM) in SSM (10 mM) did not differ significantly pre- and post-lyophilization. These data indicated that GLP-1(7-36) molecules remain associated with SSM after being lyophilized and reconstituted.

### Example 10

### GLP-1(7-36) Exhibits Anti-Inflammatory Effects In Vivo when Delivered in SSM

The purpose of this study was to determine whether GLP-1(7-36) exhibits anti-inflammatory effects in vivo when delivered in SSM. C57B6/DBA transgenic mice with NF-kappa B (NF-κB) reporter gene (HIV-LTR/luciferase (HLL)) (as described by Sadikot et al. Am. J. Respir. Cell Mol. Biol. 164: 873-8, 2001) were divided into 5 groups: untreated control (untreated control with no drug or LPS given), saline+LPS (S+LPS), SSM+LPS (M+LPS), GLP-1(7-36) in saline+LPS (G+LPS) and GLP-1-SSM+LPS (GM+LPS) [n=3 for each group]. The dose of GLP-1(7-36) was 15 nmol/mouse and that of empty nanomicelles was 0.45 μmol/mouse (at lipid concentration of 4.5 mM). Treatment protocol involved the administration of a first dose subcutaneously (s.c.) 12 h before exposure followed by second s.c. dose immediately before exposure to aerosolized LPS. All mice were sacrificed 4 h after completing LPS nebulization. Bronchoalveolar lavage (BAL) was performed and lungs were surgically removed.

For transgenic mice used in these experiments, luciferase activity in lung tissue is proportional to NF-κB activity, a pro-inflammatory transcription factor. Aerosolized LPS induced acute lung inflammation that was significantly downregulated only by nanomicellar GLP-1 (GM+SSM group). Luciferase activity in the nanomicellar GLP-1-treated group was also significantly lower than that of the GLP-1(7-36) alone-treated group (G+LPS group). Similar results were observed with myeloperoxidase (MPO) activity in lung tissue homogenates. This assay determines neutrophil enzymatic activity and is related to magnitude of neutrophilic influx into lung tissues in response to LPS. Likewise, among the LPS-exposed mice, total cell count and neutrophil count in BAL fluid were significantly lower in the nanomicellar GLP-1 group (GM+LPS) compared to saline and empty nanomicelles treatment groups.

Nanomicellar GLP-1, GLP-1 in saline (each, 15 nmol/mouse) and saline (each group, n=5) were administered s.c. to mice 30 min after completion of LPS nebulization. Nanomicellar GLP-1 significantly attenuated neutrophil cell count in BAL fluid of LPS-exposed mice compared to GLP-1 alone and saline. Taken together, these data indicate nanomicellar GLP-1 (GLP-1-SSM) is efficacious after exposure to LPS, a scenario encountered in clinical practice. Thus, GLP-1-SSM is effective in reducing inflammation.

### Example 11

### GLP-1(7-36) Exhibits Anti-Inflammatory Effects In Vitro and In Vivo when Delivered in SSM

In this study, nanomicellar GLP-1 was used to determine its effects on NF-κB activation both in vitro and in vivo. To develop a convenient, semi-quantitative method to examine NF-κB activation in vivo, a line of transgenic mice that possesses the proximal 5′ human immunodeficiency virus (HIV-1) long terminal repeat (LTR) driving the expression of Photinus luciferase cDNA [referred to as HLL mice (HIV-LTR/Luciferase)] was engineered (as described by Sadikot et al. Am. J. Respir. Cell Mol. Biol. 164: 873-8, 2001). The proximal HIV-LTR is a well-characterized NF-κB responsive promoter containing a TATA box, an enhancer region between −82 and −103 with two NF-κB motifs, and three Sp1 boxes from −46 to −78. In primary cell culture, NF-κB activation is absolutely required for transcriptional activity of HIV-LTR. Thus, these mice were used extensively to detect NF-κB activation in vivo as mouse models of acute lung inflammation and infection. The advantage of these mice is that luciferase activity in the lung is used as a surrogate marker of NF-κB activation. Accordingly, the effects of nanomicellar GLP-1 in a model of LPS-induced acute lung inflammation were determined.

Mice were treated with nanomicellar GLP-1 (3 nmol/mouse) or empty nanomicelles 12 h before exposure to aerosolized LPS (1 mg/ml) or endotoxin-free saline. Mice were harvested 4 h thereafter. BAL was performed and total and differential cell counts were determined. Lungs were homogenized for luciferase activity determination. There was a significantly lower number of neutrophils in BAL fluid of mice treated with nanomicellar GLP-1 compared to empty nanomicelles. In addition, lung luciferase activity was significantly lower in mice treated with nanomicellar GLP-1 compared to empty nanomicelles indicating that nanomicellar GLP-1 attenuates acute lung inflammation by inhibiting NF-κB activation in vivo.

To determine whether nanomicellar GLP-1 also inhibits NF-κB activity, experiments were carried out using bone marrow-derived mononuclear cells (BMDM) obtained from NF-κB reporter HLL mice. BMDM were isolated from the mice as described by Sadikot et al. (J. Immunol. 172: 1801-1808, 2004). Cells were treated with nanomicellar GLP-1 (GLP-SSM), GLP-1 in phosphate-buffered saline (GLP) (each GLP-1 dosage was 3 nmol), empty nanomicelles (SSM), or phosphate-buffered saline (PBS). Cells were then exposed to LPS (100 ng/ml) or PBS for 4 and 24 h. Luciferase activity was significantly inhibited in these BMDM cells with treatment with GLP-SSM.

### Example 12

### Activity of 17-AAG in SSM in a Model of Acute Lung Injury

17-(Allylamino)-17-demethoxygeldanamycin (17-AAG) was self-associated with SSM to see how nanomicellar 17-AAG affects Heat Shock Protein 90 (Hsp90) in the lung during acute inflammatory response to inhaled LPS. 17-AAG is an ansamycin antibiotic which binds to Hsp90 and alters it function. Hsp90 plays a key role in regulating the physiology of cells exposed to environmental stress and in maintaining the malignant phenotype of tumor cells. 17-AAG binds with a high affinity into the ATP binding pocket in Hsp90 and induces the degradation of proteins that require this chaperone for conformational maturation.

Using BMDM obtained from HLL mice as described by Sadikot et al. (supra, 2004), the efficacy of nanomicellar 17-AAG in inhibiting NF-κB activation was determined. BMDM were treated with nanomicellar 17-AAG or empty nanomicelles for 30 min and then exposed to LPS (100 ng/ml) or endotoxin-free PBS. BMDM were harvested 24 h after exposure and luciferase activity (as a surrogate marker for NF-κB activation) was measured. There was a significant inhibition of luciferase activity (i.e. NF-κB activity) in BMDM of HLL mice that were treated with nanomicellar 17-AAG 24 hours post-treatment with LPS compared to those treated with blank micelles.

Thus, 17-AAG-SSM decreases NF-κB activity indicating that 17-AAG-SSM is useful in treating NF-κB-driven inflammation and tissue injury.

### Example 13

### Activity of TREM-1 Peptide (LP17) in SSM in a Model of Acute Lung Injury

Triggering receptor expressed on myeloid cells (TREM-1) is upregulated in macrophages of mice after injection with LPS. LP17, a 17-amino acid peptide (LQVTDSGLYRCVIYHPP (SEQ ID NO: 1)) alternatively known as TREM-1 peptide or TREM-1 binding protein (T1BP), is a synthetic soluble TREM-1 decoy receptor which functions as a TREM-1 inhibitor. The objective of this study was to determine the α-helicity of LP17 in SSM and examine its biological activity in SSM in vivo.

The α-helicity of LP17 (20 μM) in saline and in SSM (5 mM) was tested. A control peptide (TDSRCVIGLYHPPLQVY (SEQ ID NO:2)) was also tested in saline and in SSM at the same concentrations. No significant difference in percent α-helicity was found when the control peptide was incubated with saline or SSM. However, significantly greater (p<0.05) α-helicity was found when LP17 was incubated with SSM as compared to saline.

The efficacy of nanomicellar LP17 was tested in a mouse model of ALI induced by aerosolized LPS. Wild-type mice were treated with LP17 or control peptide and with LP17 or control peptide self-associated with nanomicelles (each, 3 nmol; a dose similar to that used in previous experiments). Free peptides were administered subcutaneously 48 and 24 h before administration of LPS. Peptides self-associated with nanomicelles were administered to the mice only 48 h before LPS nebulization. Mice were exposed to nebulized LPS in a dose of 1 mg/ml for 40 min as previously described (Sadikot et al., Am. J. Respir. Crit. Care Med. 164: 873-8, 2001.) Control mice were treated with nebulized endotoxin-free PBS. TREM-1 was induced in lungs of mice at 4 hours after treatment with LPS. After 4 h, a bronchoalveolar lavage was carried out to determine total cell count and neutrophil count. Lungs, liver, and spleen were harvested from the mice and frozen for RNA and protein extraction. Real-time RT-PCR was carried out to analyze fold induction in TREM-1 gene expression.

The expression of TREM-1 mRNA was attenuated in mice receiving 2 doses of LP17 as compared to mice treated with control peptide. Mice that received nanomicelles with LP17 showed a significant blockade of TREM-1 compared to mice that received control nanomicelles or the naked peptide.

There was a significant reduction in total cell and neutrophil counts in mice treated with nanomicellar LP17. In separate histopathological experiments, these observations were corroborated by finding significant attenuation of lung inflammation in mice treated with nanomicellar LP17. There was a significant induction of TREM-1 expression in lungs, liver and spleen of mice that were treated with control nanomicelles and LPS, whereas mice treated with nanomicellar LP17 showed significant attenuation of TREM-1 expression in all three organs. These data indicate that nanomicellar LP17 is efficacious in blocking TREM-1 in the lung in a mouse model of acute lung inflammation. Thus, LP17-SSM is useful in treating inflammation.

### Example 14

### Sterile Filtration of GLP-1-SSM does not Affect its Biophysical Properties

GLP-1-SSM (hydrodynamic diameter, −15 nm) has shown significantly greater anti-inflammatory activity against acute lung injury (ALI) in vivo compared to free GLP-1 peptide (GLP-1(7-36) peptide amide). Because GLP-1-SSM is promising in the treatment of ALI and given that parenteral dispersion must be sterilized before clinical use, experiments were carried out to determine if GLP-1-SSM is compatible to sterile filtration through 0.2 um membrane filters.

GLP-1-SSM was prepared as described by Lim et al. (Int. J. Pharm. 356:345-350, 2008). Briefly, weighted amount of DSPE-PEG₂₀₀₀ was dissolved in saline, vortexed until complete dissolution and equilibrated for 1 hr at 25° C. in the dark. A measured volume of GLP-1 stock solution (in saline) was added to SSM dispersion to achieve the final lipid and peptide concentrations of 1 mM and 33 μM respectively followed by 2 hr incubation at 25° C.

GLP-1-SSM dispersions were filtered through a Durapore® membrane filter with tortuous pores (Millipore, Billerica, Mass.) or a Nuclepore® membrane filter with straight through pores (Whatman Inc., Piscataway, N.J.). Filtered GLP-1-SSM dispersions were analyzed by particle size analysis (7030 Nicomp DLS), quasi-elastic light scattering (QELS), circular dichroism (Jasco J-710 Spectropolarimeter; λ scan=190-260 nm), fluorescence spectroscopy (SLM Aminco 8000 spectrofluorimeter; Ex λ=275 nm), modified Bartlett phosphate assay and GLP-1 ELISA (Bachem; Cat#S-1141). Data were compared to pre-filtered control dispersions.

Filtration of GLP-1-SSM dispersion through 0.2 um membrane filters was not associated with significant changes in particle size, peptide secondary conformation and peptide-nanomicelle interaction (p>0.05; each experiment, n=3). Likewise, phospholipid content and peptide yield of filtered GLP-1-SSM dispersion were similar compared to pre-filtered dispersions (p>0.05; each experiment, n=3). Similar results were observed between GLP-1-SSM filtered through membrane filters of tortuous capillary pores (Durapore®) and straight through pores (Nuclepore®) (p>0.05; each experiment, n=3).

GLP-1-SSM dispersion showed similar particle size, associated peptide fluorescence emission and secondary conformation after filtration through 0.2 um pore size membrane filters. There was no significance loss of phospholipid and peptide content of GLP-1-SSM after sterile filtration. Therefore, GLP-1-SSM is robust to sterile filtration through 0.2 um pore size. This technique can be used for final sterilization of GLP-1-SSM dispersion for human use.

### Example 15

### VIP-SSM in Treating Ocular Infection

To determine the effect of VIP-SSM in treating ocular infection, the following experiment was carried out. Eight week old female C57BL/6 mice were given a subconjuctival injection (left eye) evoked by Pseudomonas aeruginosa (as described in Hazlett et al., J. Immunol. 179: 1138-46, 2007) that leads to perforation of the cornea if left untreated. Mice were treated topically on the eye with 5 ml containing empty micelles (control group, n=5) or VIP (5 nM) conjugated micelles (VIP-SSM) (experimental group, n=5) on day −1 (one day before infection). Mice were then routinely infected in the morning (AM) of day 0 and received a topical application (5 μl) of empty micelles or VIP-SSM in the afternoon (PM). Mice received one topical treatment, as described above, on days 1, 2, and 3, and disease grades were recorded at each time point. On day 5, experiments were terminated. Animals were euthanized and corneas were collected and stored for later isolation of mRNA and real-time PCR experiments.

The control group had 4/5 corneas with a grade of +4 (perforation) and the remaining 1/5 showed +3 grade infection with dense opacity covering the entire anterior segment and central corneal thinning. The experimental group had 1/5 that showed a +2 grade with a dense opacity covering all or part of the pupil, 2/5 that had a +3 grade (as described above), and 2/5 that showed a +4 (perforation). Grading was carried out as described by Hazlett et al. (2007, supra).

Data show a statistically significant (Mann-Whitney) reduction in eye infection (disease) in the VIP-SSM-treated corneas at 3 (p=0.02) and 5 days (p=0.01) when compared with the control group. For additional reference, see Szliter et al. (J. Immunol. 178: 1105-14, 2007). These data indicate that VIP in lipid-based formulations, like SSM or SSL, is useful in treating infections of the eye.

### Example 16

### The Use of a Combination of GLP-1(7-36)-SSM, LP17-SSM, and 17-AAG-SSM in the Treatment of Inflammation

In this study, the efficacy of a combination of GLP-1(7-36), LP17, and 17-AAG in SSM is tested to see if there is an improved or additive anti-inflammatory effect by using a combination of one or more of the above-mentioned compounds loaded into micelles.

Transgenic mice whose luciferase activity in lung tissue is proportional to NF-κB activity are treated with GLP-1(7-36)-SSM, LP17-SSM, and 17-AAG-SSM, alone and in combination (GLP-1-LP17-17-AAG-SSM) with appropriate controls, (each mouse receiving a 3 nmol dose of each, which is a dose similar to that used in previous experiments). Treatment protocol involves the administration of a first treatment dose subcutaneously (s.c.) 12 h before exposure to aerosolized LPS followed by second s.c. dose immediately before exposure to aerosolized LPS. All mice are sacrificed 4 h after completing LPS nebulization. Bronchoalveolar lavage (BAL) is performed and lungs are surgically removed.

Luciferase activity in lung tissue is measured as an indicator of NF-κB activity. Myeloperoxidase (MPO) activity in lung tissue homogenates is also measured. This assay determines neutrophil enzymatic activity and is related to magnitude of neutrophilic influx into lung tissues in response to LPS. Total cell count and neutrophil count in BAL fluid are also measured. It is expected that there is an improved effect in the treatment of inflammation with a combination of one or more of the compounds loaded in SSM.

### Example 17

### Activity of GLP-1-SSM in a Model of Hyperglycemia

Blood glucose concentration was determined in four groups of five mice each as follows: (1) untreated controls, (2) LPS-exposed, (3) LPS-exposed and GLP-1(1250 μg/kg)-treated, and (4) LPS-exposed and GLP-1(1250 μg/kg)-SSM-treated, blood glucose levels are lower in GLP-1-SSM mice treated with ALT (as discussed previously herein) than in control mice (saline control) or in mice treated with GLP-1 alone. Blood was obtained by cardiac puncture at the conclusion of the treatment. Median glucose concentration was 109, 183, 157, and 125 mg/dl, respectively. These data indicate that hyperglycemia is present in mice with LPS-induced ALI and that nanomicellar GLP-1 lowers blood glucose concentration to a greater extent than GLP-1 alone.

To determine if GLP-1-SSM has a glucose regulating effect, GLP-1 SSM is tested in an animal model with hyperglycemia (db/db (diabetic) mice; Jackson Laboratory (Bar Harbor, Me.)). Within 6 weeks of age, db/db mice develop significant obesity, fasting hyperglycemia, and hyperinsulinemia. Six db/db mice (8-12 wk) per group, are fed a controlled diet and are treated as set out in the previous experiment above. It is expected that nanomicellar GLP-1 lowers blood glucose concentration to a greater extent than GLP-1 alone in these diabetic mice.

The invention has been described in terms of particular embodiments found or proposed to comprise preferred modes for the practice of then invention. It will be appreciated by those of ordinary skill in the art that, in light of the present disclosure, numerous modifications and changes can be made in the particular embodiments exemplified without departing from the intended scope of the invention. Therefore, it is intended that the appended claims cover all such equivalent variations which come within the scope of the invention as claimed.

1-17. (canceled)

18. A sterically stabilized micelle or liposome composition comprising a water-insoluble agent, the micelle or liposome composition having a configuration that prevents aggregate formation of the agent, wherein the water-insoluble agent is glucagon-like peptide-1 (GLP-1), GLP-2, triggering receptor expressed on myeloid cells (TREM-1) peptide, TREM-2, TREM-3, or 17-(allylamino)-17-demethoxygeldanamycin (17-AAG), or a fragment or analog thereof.

19-20. (canceled)

21. The composition of claim 18, wherein the sterically stable micelle or liposome composition remains stable for at least 48 hours at room temperature.

22-24. (canceled)

25. A method of treating a condition associated with inflammation or injury in a subject comprising the step of administering to the subject the sterically stabilized micelle or liposome composition of claim 18 in an amount effective to decrease inflammation or injury.

26-27. (canceled)

28. A method of treating a condition associated with toxemia, inflammation, infection, bacteremia, sepsis, septic shock, sepsis, acute lung injury, acute respiratory distress syndrome (ARDS), severe acute respiratory syndrome (SARS), systemic inflammatory response syndrome (SIRS), or multiple organ dysfunction syndrome (MODS) in a subject comprising the step of administering to the subject the composition of claim 18 in an amount effective to treat the condition.

29-31. (canceled)

32. The method of claim 28, wherein GLP-1, GLP-2, TREM-1 peptide, TREM-2, or TREM-3 is in a D isoform, or an L isoform, or a combination of both D and L isoforms.

33. The method of claim 28, wherein the compound is linked to the sterically stabilized micelle or liposome composition.

34. (canceled)

35. The method of claim 28, wherein the inflammation or injury is of the lung or chest.

36. A method of decreasing infection, bacteremia, sepsis, or septic shock in a subject comprising the step of administering to the subject a sterically stabilized micelle or liposome composition comprising vasoactive intestinal peptide (VIP), and fragments and analogs thereof, in an amount and under conditions effective to decrease infection, bacteremia, sepsis, or septic shock, wherein the sterically stabilized micelle or liposome has configuration that prevents aggregate formation of the agent.

37. The method of claim 36, wherein the VIP is in a D isoform, or an L isoform, or a combination of both D and L isoforms.

38. (canceled)

39. A method of treating or preventing hyperglycemia in a subject comprising the step of administering to the subject a sterically stabilized micelle or liposome composition comprising GLP-1, and fragments and analogs thereof, in an amount and under conditions effective to decrease hyperglycemia, wherein the sterically stabilized micelle or liposome has configuration that prevents aggregate formation of the agent.

40. (canceled)

41. The composition of claim 18, wherein the GLP-1, GLP-2, TREM-1 peptide, TREM-2, or TREM-3 is in a D isoform, or an L isoform, or a combination of both D and L isoforms.

42. The composition of claim 18, wherein the compound is linked to the sterically stabilized micelle or liposome composition.

43. The method of claim 39, wherein the GLP-1 is in a D isoform, or an L isoform, or a combination of both D and L isoforms.

44. The method of claim 39, wherein the GLP-1 is linked to the sterically stabilized micelle or liposome composition.
